(19) United States
(12) Patent Application Publication
(10) Pub. No.: US 2008/0038269 A1
(43) Pub. Date: Feb. 14, 2008

(54) Methods for detecting and treating kidney disease

(75) Inventor: Quaggin Susan, Toronto (CA)

Correspondence Address: HAMRE, SCHUMANN, MUELLER & LARSON, P.C., P.O. BOX 2902, MINNEAPOLIS, MN, 55402-0902, US

(73) Assignee: MOUNT SINAI HOSPITAL, Toronto (CA)

(21) Appl. No.: 11/807,263

(22) Filed: May 25, 2007

Related U.S. Application Data
(60) Provisional application No. 60/808,252, filed on May 25, 2006.

(51) Int. Cl. A61K 39/00; A61K 48/00; A61P 13/12; C12Q 1/68; G01N 1/30; G01N 33/53

(52) U.S. Cl. 424/139.1; 424/93.1; 424/93.2; 435/40.5; 435/6; 435/7.1

(57) ABSTRACT

A method is provided for diagnosing and monitoring kidney disease or a predisposition to kidney disease, in a subject comprising detecting pVHL, VEGF-A, CXCR4, integrin β-1, PDGF-A, HIF1α and/or TGFβ in a sample from the subject. Screening methods for test agents for inhibiting kidney disease, and therapeutic applications are also described.

### FIELD OF THE INVENTION

The invention relates to methods for detecting and treating kidney disease.

### BACKGROUND OF THE INVENTION

Glomerular disease accounts for 20% of all ESRD in North America. The most dramatic of these is Rapid progressive glomerulonephritis (RPGN), a potentially fatal disease and one of the few diagnostic emergencies that occurs in nephrology. If left untreated, it rapidly progresses to renal failure within days to weeks. There are three major categories of RPGN based on the presence and type or absence of immune deposits. RPGN with immune deposits is classified as pauci-immune and is characterized by necrotizing glomerular vasculitis with prominent segmental fibrin deposits.

The pathogenesis of pauci-immune RPGN is incompletely understood. The identification of anti-neutrophil cytoplasmic antibodies (p- and c-ANCA) in a cohort of patients with this disease was recognized as a major breakthrough [Davies et al, 1982, Falk et al, 1990], and much of the work in this area has focused on the role of this circulating antibody and immune cells in the pathogenesis of this disease [Xiao et al, 2002].

### SUMMARY OF THE INVENTION

Novel biomarkers have been identified for diagnosis and monitoring (i.e., monitoring progression or therapeutic treatment) of kidney diseases, in particular RPGN, more particularly pauci-immune RPGN.

The markers of kidney disease include one or more hypoxia related polypeptides, including von Hippel Lindau protein (pVHL), vascular endothelial growth factor A (VEGF-A), chemokine receptor chemokine (C—X—C motif) receptor 4 (CXCR4), hypoxia inducible factor alpha (HIF-1α), integrin β-1, platelet-derived growth factor-A (PDGF-A), transforming growth factor beta (TGFβ), and Interacting Polypeptides thereof. These markers including but not limited to native-sequence polypeptides, isoforms, chimeric polypeptides, all homologs, fragments, and precursors of the markers, and modified forms of the polypeptides and derivatives are referred to herein as “Kidney Disease Marker(s)” or “KD Markers”. Polynucleotides encoding KD Markers or expressing KD Markers are referred to herein as “Kidney Disease Polynucleotide Marker(s)”, “polynucleotides encoding kidney disease marker(s)” or “KD Polynucleotides”. The KD Markers and KD Polynucleotides are sometimes collectively referred to herein as “marker(s)”.

Broadly stated, the invention provides a set of markers that can distinguish kidney diseases. In an aspect, the invention contemplates polypeptide marker sets that distinguish kidney diseases comprising or consisting essentially of at least 2, 3, 4, 5, 6 or 10 KD Markers. In an aspect the protein marker sets comprise or consist of protein clusters, or proteins in pathways comprising the KD Markers.

In another aspect, the invention provides gene marker sets that distinguish kidney diseases and uses therefor. A genetic marker set may comprise or consist essentially of a plurality of genes comprising or consisting of at least 2, 3, 4, 5, 6, 7, or 10 KD Polynucleotides. In an aspect, the gene marker sets comprise gene clusters which may be represented by dendograms, or comprise genes in pathways of KD Polynucleotides.

KD Markers and KD Polynucleotides have application in the determination of the status of kidney disease, and in particular in the detection of kidney disease or onset of kidney disease. Thus, the markers can be used for diagnosis, monitoring (i.e. monitoring progression or therapeutic treatment), prognosis, treatment, or classification of kidney disease, or as markers before or after therapy.

In accordance with an aspect of the invention, one or more of von Hippel Lindau protein (pVHL), vascular endothelial growth factor A (VEGF-A), chemokine receptor chemokine (C—X—C motif) receptor 4 (CXCR4), hypoxia inducible factor alpha (HIF-1α) transforming growth factor beta (TGFβ), integrin β-s, PDGF-A, and polynucleotides encoding the polypeptides may be used for the diagnosis, monitoring, and prognosis of kidney disease, in particular RPGN or IgA nephropathy, more particularly pauci-immune RPGN.

The levels of KD Polynucleotides and KD Markers in a sample may be determined by methods as described herein and generally known in the art. The expression levels may be determined by isolating and determining the level of nucleic acid transcribed from each KD Polynucleotide. Alternatively or additionally, the levels of KD Markers may be determined.

In accordance with methods of the invention, susceptibility to kidney disease can be assessed or characterized, for example by detecting or identifying the presence in the sample of (a) a KD Marker or fragment thereof; (b) a metabolite which is produced directly or indirectly by a KD Marker; (c) a transcribed polynucleotide or fragment thereof having at least a portion with which a KD Polynucleotide is substantially identical; and/or (c) a transcribed polynucleotide or fragment thereof, wherein the polynucleotide hybridizes with a KD Polynucleotide.

In an aspect, a method is provided for characterizing susceptibility to kidney disease by detecting one or more KD Markers or KD Polynucleotides in a subject comprising:
(a) obtaining a sample from a subject;
(b) detecting or identifying in the sample KD Markers and/or KD Polynucleotides; and
(c) comparing the detected amount with an amount detected for a standard.

In a particular aspect of the invention, a method is provided for detecting one or more KD Markers and/or KD Polynucleotides in a subject or for diagnosing or monitoring kidney disease in a subject comprising:

(a) obtaining a sample from a patient;

(b) detecting in the sample KD Markers and/or KD Polynucleotides; and

(c) comparing the detected amount with an amount detected for a standard.

The term “detect” or “detecting” includes assaying, imaging or otherwise establishing the presence or absence of the target KD Polypeptides or KD Polynucleotides encoding the markers, subunits thereof, or combinations of reagent bound targets, and the like, or assaying for, imaging, ascertaining, establishing, or otherwise determining one or more factual characteristics of kidney disease or similar conditions. The term encompasses diagnostic, prognostic, and monitoring applications for the KD Markers and KD Polynucleotides.

The invention also provides a method of assessing whether a patient has kidney disease or a pre-disposition for kidney disease comprising comparing:
(a) levels of one or more KD Markers and KD Polynucleotides in a sample from the patient; and
(b) normal levels of one or more KD Markers and KD Polynucleotides in samples of the same type obtained from control patients, wherein altered levels of the KD Markers or KD Polynucleotides relative to the corresponding normal levels of the markers or polynucleotides is an indication that the patient has kidney disease or has a predisposition to kidney disease.

In an aspect of a method of the invention for assessing whether a patient has kidney disease or a pre-disposition for kidney disease, higher levels of KD Markers or KD Polynucleotides in a sample relative to the corresponding normal levels is an indication that the patient has kidney disease or a pre-disposition for kidney disease.

In another aspect of a method of the invention for assessing whether a patient has kidney disease or a pre-disposition for kidney disease, lower levels of KD Markers or KD Polynucleotides in a sample relative to the corresponding normal levels is an indication that the patient has kidney disease or a pre-disposition for kidney disease.

In a further aspect of the invention, a method for screening or monitoring a subject for kidney disease is provided comprising (a) obtaining a biological sample from a subject; (b) detecting the amount of one or more KD Markers and KD Polynucleotides associated with kidney disease in said sample; and (c) comparing said amount of KD Markers and KD Polynucleotides detected to a predetermined standard, where detection of a level of KD Markers and KD Polynucleotides that differs significantly from the standard indicates kidney disease or onset of kidney disease.

A significant difference between the levels of a KD Marker or KD Polynucleotide in a patient and the normal levels is an indication that the patient has kidney disease or a predisposition to kidney disease.

In an embodiment the amount of KD Marker(s) or KD Polynucleotide(s) detected is greater than that of a standard (e.g., VEGF-A, CXCR4, INTEGRIN B-1, PDGF-A, HIF1α, HIF2α, and TGFβ) and is indicative of kidney disease. In another embodiment the amount of KD Marker(s) or KD Polynucleotide(s) detected is lower than that of a standard (pVHL) and is indicative of kidney disease or onset of kidney disease.

A method of diagnosing or monitoring kidney disease or onset of kidney disease in a subject is provided comprising obtaining a biological sample from the subject, identifying KD Polypeptides and KD Polynucleotides in the sample associated with kidney disease to identify kidney disease of a particular etiology, and providing an individualized therapeutic strategy based on the etiology of kidney disease identified.

In one aspect the invention provides a method for determining kidney disease development potential in a patient at risk for the development of kidney disease comprising the steps of determining the concentration of one or more KD Marker comprising or selected from the group consisting of pVHL, VEGF-A, CXCR4, integrin β-1, PDGF-A, HIF1α, HIF2α, TGFβ, and Interacting Polypeptides, or KD Polynucleotides encoding same, in a sample (e.g. serum or plasma) from the patient, comparing the concentration of the markers to a cut-off concentration and determining kidney disease development potential from the comparison, wherein concentrations of markers above the cut-off concentration are predictive of (e.g., correlate with) kidney disease development in the patient.

In aspects of the methods of the invention, the methods are non-invasive for detecting kidney disease, which in turn allow for diagnosis of a variety of conditions or diseases associated with such kidney disease.

In particular, the invention provides a non-invasive non-surgical method for detection, diagnosis, monitoring, or prediction of kidney disease or onset of kidney disease in a patient comprising: obtaining a sample of blood, plasma, serum, urine or saliva or a tissue sample from the patient; subjecting the sample to a procedure to detect one or more KD Marker(s) or KD Polynucleotide(s) in the blood, plasma, serum, urine, saliva or tissue; detecting, diagnosing, and predicting kidney disease by comparing the levels of KD Marker(s) or KD Polynucleotide(s) to the levels of KD Marker(s) or KD Polynucleotide(s) obtained from a control.

In an embodiment, kidney disease or onset of kidney disease is detected, diagnosed, or predicted by determination of decreased levels of markers when compared to such levels obtained from a control.

In another embodiment, kidney disease or onset of kidney disease is detected, diagnosed, or predicted by determination of increased levels of markers when compared to such levels obtained from the control.

The invention provides a method for monitoring the progression of kidney disease in a patient the method comprising:
(a) detecting one or more KD Markers or KD Polynucleotides in a sample from the patient at a first time point;
(b) repeating step (a) at a subsequent point in time; and
(c) comparing the levels detected in (a) and (b), and therefrom monitoring the progression of the kidney disease.

The invention also provides a method for assessing the potential efficacy of a test agent for preventing, inhibiting, or reducing kidney disease or onset of kidney disease, and a method of selecting an agent for preventing, inhibiting or reducing kidney disease.

The invention also contemplates a method of assessing the potential of a test compound to contribute to kidney disease or onset of kidney disease comprising:
(a) maintaining separate aliquots of tissue from a patient in the presence and absence of the test compound; and
(b) comparing the levels of one or more of KD Markers and KD Polynucleotides in each of the aliquots.

A significant difference between the levels of one or more KD Markers or KD Polynucleotides in an aliquot maintained in the presence of (or exposed to) the test compound relative to the aliquot maintained in the absence of the test compound, indicates that the test compound potentially contributes to kidney disease or onset of kidney disease.

The invention also provides a method for determining the effect of an environmental factor on kidney disease comprising comparing one or more KD Markers or KD Polynucleotides associated with kidney disease or onset of kidney disease in the presence and absence of the environmental factor.

The invention further relates to a method of assessing the efficacy of a therapy for preventing, inhibiting, or reducing kidney disease or onset of kidney disease in a patient. A method of the invention comprises comparing: (a) levels of one or more KD Markers and KD Polynucleotides in a sample from the patient obtained from the patient prior to providing at least a portion of a therapy to the patient; and (b) levels of the KD Markers and/or KD Polynucleotides in a second sample obtained from the patient following therapy.

A significant difference between the levels of KD Markers and/or KD Polynucleotides in the second sample relative to the first sample is an indication that the therapy is efficacious for inhibiting kidney disease or onset of kidney disease.

In an embodiment, the method is used to assess the efficacy of a therapy for inhibiting kidney disease or onset of kidney disease, where lower levels of KD Markers and/or KD Polynucleotides (e.g. VEGF-A, VEGF-A, CXCR4, integrin β-1, PDGF-A, HIF1α, HIF2α, and TGFβ) relative to the first sample, is an indication that the therapy is efficacious for inhibiting the disease.

In an embodiment, the method is used to assess the efficacy of a therapy for inhibiting kidney disease or onset of kidney disease, where higher levels of KD Markers and/or KD Polynucleotides (e.g. pVHL) relative to the first sample, is an indication that the therapy is efficacious for inhibiting kidney disease or onset of kidney disease.

The “therapy” may be any therapy for treating kidney disease or onset of kidney disease in particular, including but not limited to therapeutics, and procedures and interventions. A method of the invention can be used to evaluate a patient before, during, and after therapy.

Certain methods of the invention employ one or more polynucleotides capable of hybridizing to one or more KD Polynucleotides. Thus, methods for monitoring kidney disease or onset of kidney disease are contemplated comprising detecting KD Polynucleotides associated with kidney disease.

The invention relates to a method of characterizing a biological sample by detecting or quantitating in the sample one or more KD Polynucleotides extracted from the sample that are characteristic of kidney disease the method comprising assaying for differential expression of KD polynucleotides in the sample. Differential expression of the polynucleotides can be determined by micro-array, hybridization or by amplification of the extracted polynucleotides.

The invention contemplates a gene expression “signature” comprising KD Polynculeotides that is associated with kidney disease. This signature provides a highly sensitive and specific test with both high positive and negative predictive values permitting diagnosis and prediction of kidney disease.

The present invention relates to a method for diagnosing and monitoring kidney disease or onset of kidney disease in a sample from a subject comprising isolating polynucleotides, in particular mRNA, from the sample; and detecting KD Polynucleotides in the sample. The presence of different levels of KD Polynucleotides in the sample compared to a standard or control may be indicative of kidney disease, stage of kidney disease, onset of kidney disease, and/or a positive prognosis.

In an embodiment of the invention, KD Polynucleotide positive samples (e.g. higher levels of selected KD Polynucleotides compared to a normal control) are a negative diagnostic indicator. Positive samples can be indicative of kidney disease, advanced kidney disease, onset of kidney disease, or a poor prognosis.

In another embodiment of the invention, KD Polynucleotide negative samples (e.g. lower levels of selected KD Polynucleotides compared to a normal control) are a negative diagnostic indicator. Negative samples can be indicative of kidney disease, advanced kidney disease, onset of kidney disease, or poor prognosis.

In an aspect, the invention provides a method for characterizing or classifying a sample as associated with kidney disease comprising detecting a difference in the expression of one or more KD Polynucleotides, in particular genes encoding pVHL, VEGF-A, VEGF-A, CXCR4, integrin β-1, PDGF-A, HIF1α and TGFβ, relative to a control. A method may comprise detecting a plurality of KD Polynucleotides or genes comprising at least 2, 3, 4, 5, 6, 7, 10, 15, 25, 30, 50, 75 or 100 genes. In a particular aspect, the control comprises polynucleotides derived from a pool of samples from normal patients.

The invention provides methods for determining the presence or absence of kidney disease in a subject comprising detecting in the sample levels of polynucleotides that hybridize to one or more KD Polynucleotides, comparing the levels with a predetermined standard or cut-off value, and therefrom determining the presence or absence of kidney disease in the subject. In an embodiment, the invention provides methods for determining the presence or absence of kidney disease in a subject comprising (a) contacting a sample obtained from the subject with oligonucleotides that hybridize to one or more KD Polynucleotides; and (b) detecting in the sample a level of polynucleotides that hybridize to the KD Polynucleotides relative to a predetermined cut-off value, and therefrom determining the presence or absence of kidney disease in the subject.

Within certain embodiments, the amount of polynucleotides that are mRNA are detected via polymerase chain reaction using, for example, oligonucleotide primers that hybridize to one or more KD Polynucleotides, or complements of such polynucleotides. Within other embodiments, the amount of mRNA is detected using a hybridization technique, employing oligonucleotide probes that hybridize to one or more KD Polynucleotides, or complements thereof.

When using mRNA detection, the method may be carried out by combining isolated mRNA with reagents to convert to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of one or more KD Polynucleotides in the sample. For mRNA the analyzing step may be accomplished using Northern Blot analysis to detect the presence of KD Polynucleotides. The analysis step may be further accomplished by quantitatively detecting the presence of KD Polynucleotides in the amplification product, and comparing the quantity of KD Marker detected against a panel of expected values for the known presence or absence of the KD Markers in normal tissue derived using similar primers.

The invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to one or more KD Polynucleotides to produce amplification products; (d) analyzing the amplification products to detect an amount of KD Polynucleotide mRNA; and (e) comparing the amount of mRNA to an amount detected against a panel of expected values for normal tissue derived using similar nucleic acid primers.

In particular aspects of the invention, the methods described herein utilize one or more KD Polynucleotides placed on a micro-array so that the expression status of each of the markers is assessed simultaneously.

In an embodiment, the invention provides a kidney disease micro-array comprising a defined set of genes whose expression is significantly altered by kidney disease. The invention further relates to the use of the micro-array as a prognostic tool to predict kidney disease. In an embodiment, the micro-array discriminates between kidney diseases resulting from different etiologies.

In an embodiment, the invention provides for oligonucleotide arrays comprising marker sets of KD Polynucleotides. The microarrays provided by the present invention may comprise probes to markers able to distinguish kidney disease. In particular, the invention provides oligonucleotide arrays comprising probes to a subset or subsets of at least 2, 3, 4, 5, 6, 7, 8, 9, 10, 15, 20, 25, 30, 35, 40, 45, 50, 60, 70, 80, 90, 100, 150, 200, 250, or 300 gene markers up to a full set of markers which distinguish kidney disease patients or samples.

Kidney diseases may be assessed by determining the levels of KD Markers.

In an aspect the invention provides a method for detecting one or more of pVHL, VEGF-A, CXCR4, integrin β-1, PDGF-A, HIF1α, and TGFβ, comprising (a) obtaining a sample from a patient; (b) detecting or identifying in the sample one or more of pVHL, VEGF-A, CXCR4, integrin β-1, PDGF-A, HIF1α, and TGFβ; and (c) comparing the detected amount with an amount detected for a standard.

In another aspect, a method for screening a subject for kidney disease is provided comprising (a) obtaining a biological sample from a subject; (b) detecting the amount of one or more of pVHL, VEGF-A, CXCR4, integrin β-1, PDGF-A, HIF1α and TGFβ in the sample; and (c) comparing said amount of pVHL, VEGF-A, CXCR4, integrin β1, PDGF-A, HIF1α and TGFβ detected to predetermined standards, where detection of a level of one or more of pVHL, VEGF-A, CXCR4, integrin β-1, PDGF-A, HIF1α, HIF2α, and TGFβ, which is significantly different than that of a standard indicates presence or susceptibility to kidney disease.

According to a method involving one or more of pVHL, VEGF-A, CXCR4, integrin β-1, PDGF-A, HIF1α, and TGFβ that bind same, the levels in a sample from a patient are compared with the normal levels of the protein(s) in samples of the same type obtained from controls (e.g. samples from individuals not afflicted with disease). Significantly altered levels in the sample of a protein(s) relative to the normal levels in a control is indicative of a disorder, in particular a kidney disorder.

In a particular aspect, a method is provided for screening a subject for a kidney disorder, in particular RPGN, wherein a reduction or loss of pVHL and an increase in one or more of VEGF-A, CXCR4, integrin β-1, PDGF-A, HIF1α, and TGFβ, compared to a standard is indicative of the kidney disorder.

In an embodiment, the invention provides a method of assessing whether a patient is afflicted with or has a pre-disposition to kidney disease (e.g. RPGN) wherein a significant difference between the levels of one or more of pVHL, VEGF-A, CXCR4, integrin β-1, PDGF-A, HIF1α, and TGFβ in the patient and normal levels is an indication that the patient is afflicted with or has a predisposition to kidney disease. In a particular embodiment, a reduction in pVHL levels and an increase in levels of one or more of VEGF-A, CXCR4, integrin β-1, PDGF-A, HIF1α, and TGFβ compared to normal levels of the protein(s) may be indicative of RPGN, in particular pauci-immune RPGN. In another particular embodiment, a reduction in levels of one or more of VEGF-A, CXCR4, integrin β-1, PDGF-A, HIF1α, and TGFβ compared to normal levels of the protein(s) may be indicative of IgA nephropathy.

The invention provides a method of characterizing or classifying a sample by detecting or quantitating in the sample one or more KD Markers extracted from the sample that are characteristic of kidney disease, the method comprising assaying for differential expression of KD Polypeptides in the sample. Differential expression of KD Markers can be assayed using procedures known in the art, including without limitation, separation techniques known in the art, antibody microarrays, or mass spectroscopy of polypeptides extracted from a sample.

Certain methods of the invention employ binding agents (e.g. antibodies) that specifically recognize KD Markers. Therefore, in an aspect, the invention provides methods for determining the presence or absence of kidney disease or onset of kidney disease, in a patient, comprising the steps of (a) contacting a biological sample obtained from a patient with one or more binding agent that specifically binds to KD Marker(s); and (b) detecting in the sample an amount of KD Marker(s) that bind to the binding agent, relative to a predetermined standard or cut-off value, and therefrom determining the presence or absence of kidney disease in the patient.

In an aspect, the invention relates to a method for diagnosing and monitoring kidney disease in a subject by quantitating one or more KD Markers associated with kidney disease in a biological sample from the subject comprising (a) reacting the biological sample with one or more binding agent specific for the KD Marker(s) (e.g. an antibody) that are directly or indirectly labelled with a detectable substance; and (b) detecting the detectable substance.

In another aspect the invention provides a method for using an antibody to detect expression of one or more KD Markers in a sample, the method comprising: (a) combining antibodies specific for one or more KD Markers with a sample under conditions which allow the formation of antibody:marker complexes; and (b) detecting complex formation, wherein complex formation indicates expression of the markers in the sample. Expression may be compared with standards and is diagnostic of kidney disease.

KD Markers levels can be determined by constructing an antibody microarray in which binding sites comprise immobilized, preferably monoclonal, antibodies specific to a substantial fraction of marker-derived proteins of interest.

The invention also relates to kits for carrying out the methods of the invention. In an embodiment, the kit is for assessing whether a patient is afflicted with a kidney disease and it comprises reagents for assessing one or more KD Markers or KD Polynucleotides. In another embodiment, the invention provides diagnostic tools, and kits for detecting, diagnosing, and predicting the presence or onset of kidney disease by monitoring levels of one or more of KD Markers and KD Polynucleotides.

The invention further provides kits comprising the marker sets described herein. In an aspect the kit contains a micro-array ready for hybridization to target KD Polynucleotides, plus software for the data analyses.

The invention also provides a diagnostic composition comprising one or more KD Marker or a KD Polynucleotide. A composition is also provided comprising a probe that specifically hybridizes to a KD Polynucleotide, or a fragment thereof, or a binding agent (e.g., an antibody) specific for a KD Marker or a fragment thereof. In another aspect, a composition is provided comprising one or more KD Polynucleotide specific primer pairs capable of amplifying KD Polynucleotides using polymerase chain reaction methodologies. The probes, primers or binding agents (e.g., antibodies) can be labeled with a detectable substance.

Still further the invention relates to therapeutic applications for kidney disease employing KD Markers and KD Polynucleotides, and/or agonists and antagonists of the markers.

In an aspect, the invention relates to pharmaceutical compositions comprising KD Markers or parts thereof associated with kidney disease, agonists or antagonists of KD Markers associated with kidney disease, and a pharmaceutically acceptable carrier, excipient, or diluent.

The invention provides a method of treating or preventing kidney disease or onset of kidney disease in a subject afflicted with or at risk of developing kidney disease comprising administering to the subject an effective amount of an agonist of a down-regulated KD Marker or KD Polynucleotide (e.g. pVHL).

The invention provides a method of treating or preventing kidney disease or onset of kidney disease in a subject having or at risk of developing kidney disease comprising administering to the subject an effective amount of an antagonist of an up-regulated KD Marker or KD Polynucleotide (e.g. VEGF-A, CXCR4, integrin β-1, PDGF-A, HIF1α, and TGFβ.

In an aspect the invention provides a method of treating a subject afflicted with or at risk of developing kidney disease comprising inhibiting expression of one or more KD Marker or KD Polynucleotide, in particular VEGF-A, CXCR4, integrin β-1, PDGF-A, HIF1α, and TGFβ.

In another aspect, the invention provides antibodies specific for KD Markers associated with kidney disease that can be used to inhibit KD Marker or KD Polynucleotide expression.

A method for treating or preventing kidney disease or onset of kidney disease in a subject is provided comprising administering to a subject in need thereof antibodies specific for one or more up-regulated KD Markers, in particular antibodies for VEGF-A, CXCR4, integrin β-1, PDGF-A, HIF1α, and TGFβ.

The invention contemplates a method of using antagonists or agonists of KD Markers or KD Polynucleotides or parts thereof in the preparation or manufacture of a medicament for the prevention or treatment of kidney disease.

In an aspect the invention contemplates a method of using KD Markers or parts thereof, antibodies specific for KD Markers, or inhibitor of KD Polynucleotides (e.g. antisense) in the preparation or manufacture of a medicament for the prevention or treatment of kidney disease or onset of kidney disease.

The invention also provides a method for stimulating or enhancing in a subject production of antibodies directed against one or more up-regulated KD Marker (e.g. VEGF-A, CXCR4, integrin β-1, PDGF-A, HIF1α, and TGFβ). The method comprises administering to the subject one or more up-regulated KD Marker, peptides derived therefrom, or chemically produced (synthetic) peptides, or any combination of these molecules of the invention in a dose effective for stimulating or enhancing production of the antibodies.

The invention contemplates the methods, compositions, and kits described herein using additional markers associated with kidney disease. The methods described herein may be modified by including reagents to detect the additional markers, or polynucleotides for the markers.

In embodiments of the invention the methods, compositions and kits use one or more of the markers comprising pVHL, VEGF-A, CXCR4, integrin β-1, PDGF-A, HIF1α and TGFβ (e.g. SEQ ID Nos. 1). In another embodiment, they use a panel of markers selected from the markers in SEQ ID Nos. 1 through, in particular a panel comprising two or more of pVHL, VEGF-A, CXCR4, integrin β-1, PDGF-A, HIF1α, and TGFβ.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1 Selective deletion of VHL from podocytes leads to RPGN a. At three weeks of age, glomeruli from VHL^{flox/flox}/Cre (−/−) mice have dilated capillary loops (arrows). By four weeks of age, glomeruli (G) from mutant mice have cellular crescents (Cr), fibrinoid necrosis (Ne) and renal tubules (t) packed with pink proteinaceous material. The glomerulus in the left bottom panel also shows periglomerular monocytic infiltrate. C=control; b. SDS PAGE gel. 2 μl of urine was loaded in each of the lanes from control (C) or mutant VHL^{flox/flox}/Cre (−/−) mice. Note the large amount of albuminuria (66 kDa) in the mutant. MW=molecular weight c. Serum creatinine values increased 2-fold by 4 weeks of age in mutant mice compared to control littermates and indicate renal failure. d. Immunostaining for fibrin demonstrates segmental staining in all glomeruli from VHL^{flox/flox}/Pod-Cre mice (VHL mutant) that is absent from wildtype controls. Magnification: ×1500.

FIG. 2: Podocyte proliferation initiates crescent formation (a-f) Laser capture microdissection (LCM) and Lineage Tagging Shows Crescentic Cells Derive from Podocytes. a. A cellular crescent is shown by the arrow. b. Outline of crescent captured by LCM. c. Following capture, the cellular crescent has been removed from the glomerulus. d. Isolated cellular crescent. e. Genomic DNA was isolated from the crescents (Cr), whole glomeruli (G) or tubules (T) and PCR analysis performed to detect the floxed allele and/or the excised floxed allele. Excision of the floxed allele (1-loxP allele) only occurs in cells that express the Cre transgene i.e. podocytes. Cellular crescents show a single 1-loxP band demonstrating that the origin of these cells is from podocytes. In contrast, tubular cells exhibit only the 2-loxP band while glomeruli that contain podocyte and non-podocyte cell types, exhibit both bands. f. Mice were generated that carried one or both VHL floxed alleles, the Cre transgene and the Z/EG reporter transgene [Moeller et al, 2004]. Cre-mediated DNA excision occurs only within podocytes leading to expression of GFP (brown cells). VHL heterozygotes (VHL^{flox/+/}Cre/Z/EG) show expression in healthy-appearing podocytes; expression in VHL knock-outs (VHL^{flox/flox}/Cre/Z/EG) is seen in cells that populate the glomerular crescent confirming that these cells originate from the podocyte cell lineage. g. At three weeks, cells in the glomeruli (white arrowheads) of all mutant (−/−) mice are proliferating as shown by BrdU labeling (red label). Unstained glomeruli are seen in control (+/+) kidneys. Magnification: 800-1500×. h. Double immunostaining for a podocyte-restricted marker, the zonula occludens protein (ZO-1, green label), and BrdU confirm that podocytes are proliferating (yellow cells/white arrowheads). Magnification: 1500×. i. Staining for PCNA at four weeks of age confirms that cells within the crescents are proliferating (black arrowheads). Parietal epithelial (pa) cells are also proliferating at this stage. Magnification: 1800×. +/+=wildtype; −/−=VHL^{flox/flox}/Cre

FIG. 3: Increase in glomerular CXCR4 is functionally important for the RPGN phenotype. a. At four weeks of age, CXCR4 is upregulated in podocytes from mutant (−/−) mice (black arrows). Magnification: 1500×. b. Relative expression of CXCR4 in glomeruli and tubules of VHL knockout mice. Real-time PCR was used to quantify CXCR4 expression using the delta delta CT calculation, normalized to 18S and relative to control littermates. Data shown represents the mean +/− the SEM, n=3. c. The bar graph shows that at four weeks of age, mutant mice treated with anti-CXCR4 had significantly lower proteinuria than PBS-treated littermates. Similarly, the degree of hematuria was reduced in treated animals. Values shown are averages for urinary dipstick values. Mutant−VHL^{flox/flox}/Cre (+) genotype; Control=VHL^{flox/+}/Cre (+) littermates. d. At 7.5 weeks of age, 100% of mutant mice (VHL^{flox/flox}/Pod-Cre) treated with PBS vehicle showed global glomerulosclerosis (scarring of glomeruli) and succumbed to renal failure. In contrast, all mutant littermates treated with anti-CXCR4 were alive and showed a range of glomerular phenotypes including glomeruli with no sclerosis or crescents. Lower power images (top panel) demonstrate a marked difference between the degree of proteinuria and tubular dilation in treated vs. untreated mice (arrows). Magnification: 250×; 1500×. e. Model for RPGN in VHL mutant mice. Loss of VHL from the podocyte leads to stabilization of HIFs and induction of downstream targets including CXCR4. This allows podocytes (light blue) to re-enter the cell cycle, initiating crescent formation. Concurrently, the adjacent endothelium (en) is activated (pink arrow) through upregulation of cytokines such as VEGF and TNFα.

FIG. 4: De novo expression of CXCR4 in podocytes leads to proliferation and glomerular disease in mice and is found in patients with RPGN. a) Graph showing the results of the MTT proliferation assay. b) Immunostaining for CXCR4 at 2 weeks of age demonstrates podocyte-selective (po) expression in transgenic (Tg) mice. (top row) De novo expression of CXCR4 in podocytes leads to proliferative glomerular disease in transgenic mice. Glomeruli (shown at the same magnification) are markedly enlarged compared to wildtype littermates and some glomeruli show focal crescents or crescent-like structures in Bowman's space (arrow). c) Mice pulsed with BrdU. A glomerulus from a mutant CXCR4 mouse (top) shows numerous proliferating cells compared to none in controls (not shown). Podocytes that stain for BrdU are shown by the arrows (middle and bottom). Bottom 2 glomeruli are counterstained with hematoxylin. c: Immunohistochemistry for CXCR4 shows a marked increase in expression in representative glomeruli from 2 patients with pauci-immune RPGN compared to a patient with a non-crescentic glomerulopathy (MePGN). Expression of synaptopodin, a marker of differentiated podocytes, is decreased in RPGN. A few differentiated podocytes remain within crescents (white arrows and inset panels) that express both synaptopodin and CXCR4. A glomerulus from a patient with lupus nephritis (SLE) is shown as a positive control where CXCR4 expression is upregulated predominantly in glomerular endothelial cells. Light micrographs (LM) are shown from the same patient for comparison. MePGN=mesangial proliferative GN; RPGN=cANCA+RPGN; SLE=Stage III systemic lupus nephritis. Cr=crescent. Tri=trichrome; H&E=hematoxylin and eosin Magnifications: approx. 600×

FIG. 5. a. Generation of podocyte-specific VHL knockout mice. Podocin-Cre mice were bred to mice homozygous for a floxed VHL allele. Cre-mediated excision leads to loss of the promoter and 1^{st} exon of VHL leading to a null allele. b. Genotypic analysis. The Cre transgene was detected by PCR analysis; the Cre transgene from positive mice produce a band that measures approximately 300-bp. The VHL floxed allele was detected by PCR analysis; the wildtype allele measures 915-bp and the floxed allele measures 945-bp. * indicates homozygous for the floxed allele (VHL^{flox/flox}). c. Generation of a podocyte-selective CXCR4 transgene. A 2,5-kb full length coding cDNA for CXCR4 was subcloned downstream of the podocyte-selective 4.125-kb murine nephrin promoter. PA=polyA d. Genotypic analysis. The CXCR4 transgene was detected by PCR analysis and measures 281-bp.

FIG. 6. Hif1-α protein and target genes are increased in glomeruli from VHL^{flox/flox}/Cre mice and patients with RPGN. a. Immunostaining shows nuclear staining of the Hif1-α subunit in podocytes (arrows) of mutant mice but no staining in Cre-negative control littermates. Magnification ×1000. b. Table of induction of HIF1-α and HIF target genes in glomeruli from mutant mice and patients with RPGN. Mouse values were determined from microarray comparison and human values from real-time PCR. All values are reported as fold increase; *p<0.05; a,b: p<0.05 compared to IgA or control, respectively IgA=IgA non-crescentic glomerulonephritis; control were normal glomeruli obtained from renal cancer specimens.

FIG. 7. Expression of the HIF target gene, VEGF-A in glomeruli from mutant mice. At six days, glomeruli from mutant mice show an increase in the HIF target gene, VEGF-A expression in podocytes of capillary-loop stage glomeruli while another podocyte-specific marker, nephrin, is unchanged. At four weeks of age, glomeruli from mutant mice show marked upregulation of VEGF-A compared to control, and cells expressing VEGF-A can be seen within the crescent (arrowhead) and in the lumen of adjacent tubules (arrow). In contrast, nephrin is decreased in the mutant glomeruli consistent with loss of podocyte differentiation that occurs in glomerular injury. Magnification: top left: 400×; rest: 800×

FIG. 8. mRNA expression analysis of VHL and Hif target genes in glomeruli from patients with pauci-immune cANCA+RPGN, IgA nephritis or no disease. Each cluster represents an individual patient and each bar represents a separate gene. Gene expression was determined in glomeruli isolated from patient biopsy samples by realtime PCR, normalized to GAPDH, and expressed as a ratio to the mean of controls (mean of controls=1.00). Of note, VHL targets such as Hif1-α, VEGF-A, PDGF-A, TGF-β and integrin β-1 are increased in 7 of 9 patients with RPGN while they are decreased in the majority of patients with IgA nephropathy. The lack of induction of expression in 2 of the 9 ANCA+patients may be explained by the mild and focal nature of their disease (<30% of glomeruli affected) and normal renal function (serum Creatinine 0.7-1.2 mg/dL).

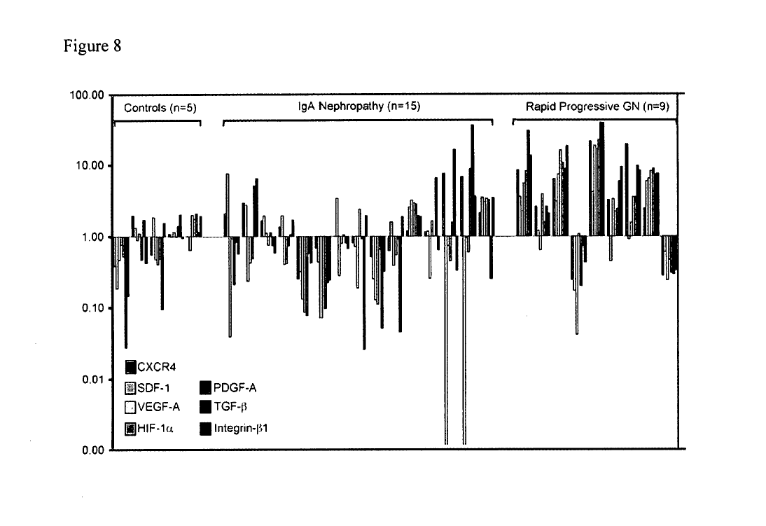

### DETAILED DESCRIPTION OF THE INVENTION

The invention relates to newly discovered correlations between expression of KD Polypeptides and KD Polynucleotides and kidney diseases. Methods are provided for detecting the presence of kidney disease in a sample, the absence of kidney disease in a sample, assessing the histology of tissues associated with kidney disease, and other characteristics of kidney disease that are relevant to prevention, diagnosis, prognosis, characterization, and therapy of kidney disease in a patient. Methods are also provided for assessing the efficacy of one or more test agents for modulating KD Polypeptides and KD Polynucleotides that affect kidney disease, assessing the efficacy of a therapy for kidney disease, monitoring the progression of kidney disease, selecting an agent or therapy for inhibiting kidney disease, treating a patient afflicted with kidney disease, inhibiting kidney disease in a patient, and assessing the potential of a test compound to contribute to kidney disease.

### GLOSSARY

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Unless defined otherwise, all technical and scientific terms used herein have the same meaning as commonly understood by one of ordinary skill in the art to which this invention belongs.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition containing “an agonist” includes a mixture of two or more agonists.

The terms “administering” or “administration” refers to the process by which a therapeutically effective amount of one or more therapeutic is delivered to a patient for treatment purposes. A therapeutic is administered in accordance with good medical practices taking into account the patient's clinical condition, the site and method of administration, dosage, patient age, sex, body weight, and other factors known to physicians.

“Agonist” refers to an agent that mimics or upregulates (e.g. potentiates or supplements) at least one KD Marker (e.g. pVHL) activity, in particular a biological and/or immunological activity of a KD Marker. An agonist can include any agent that results in activation, enhancement or alteration of the presence of a down-regulated KD Marker or KD Polynucleotide. An agonist can be a native KD Marker or derivative thereof having at least one biological activity of a native KD Marker. An agonist can be a compound that up-regulates expression of a KD Polynucleotide or which increases at least an activity of a KD Marker. An agonist can also be a compound that increases the interaction of a KD Marker and another molecule (e.g. Interacting Polypeptide). Agonists include molecules that bind to a KD Marker.

“Antagonist” or “inhibitor” refers to an agent that down-regulates (e.g. suppresses or inhibits) at least one KD Marker (e.g. CXCR4) activity, in particular a biological and/or immunological activity of a KD Marker. Antagonism can include any mechanism or treatment that results in inhibition, inactivation, blocking or reduction or alteration of the presence of an up-regulated KD Marker or KD Polynucleotide. An antagonist can be a compound that inhibits or decreases the interaction between a KD Polypeptide and another molecule (e.g., an Interacting Polypeptide). An antagonist can also be a compound that down-regulates expression of a KD Polynucleotide or which reduces the amount of a KD Marker. An antagonist can be an antisense KD Polynucleotide, siRNA, or a ribozyme capable of interacting specifically with a KD Polynucleotide RNA. Other antagonists are molecules that bind to a KD Marker and inhibit its activity, and binding agents (e.g., antibodies) interacting specifically with an epitope of a KD Marker. An antagonist can be a small molecule capable of inhibiting the interaction between a KD Marker and an Interacting Polypeptide. Examples of antagonists are antibodies specific for KD Markers, binding agents for KD Markers, and inhibitors of KD Polynucleotides (e.g. antisense).

Agonists and antogonists may be peptides such as soluble peptides including Ig-tailed fusion peptides, members of random peptide libraries and combinatorial chemistry-derived molecular libraries made of D- and/or L-configuration amino acids, carbohydrates, nucleic acids, antisense molecules, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. They may be an endogenous physiological compound or natural or synthetic compounds.

In aspects of the invention, an antagonist of CXCR4 is employed. Examples of CXCR4 antagonists include Mozobil (plerixafor) (AnorMED Inc.), AMD-070 (AnorMED Inc.), BKT140 (Biokine Therapeutics Inc.), CXCR4 monoclonal antibody (Northwest Biotherapeutics Inc.), KRH-2731/CS-3955 (Daiichi Sankyo Company), AVR 118 (reticulose) (Advanced Viral Research Corp.), CXCR4 antagonist (TaiGen Biotechnology), and CTCE-0214 (Chemokine Therapeutics Corp).

“Antisense” refers to an oligonucleotide sequence that is partially or completely complementary to a KD Polynucleotide sequence (e.g. a CXCR4 Polynucleotide sequence) (translated and untranslated regions). An antisense molecule combines with natural sequences produced in a cell to form duplexes that block either the further transcription or translation. The antisense molecules can be DNA or RNA or chimeric mixtures or derivatives or modifications thereof, single-stranded or double-stranded. The molecules can be modified at the base moiety, sugar moiety, or phosphate backbone, for example, to improve stability of the molecule, hybridization, etc. Other groups can be appended or conjugated to the molecules including peptides (e.g., for targeting host cell receptors), agents facilitating transport across the cell membranes or the blood-brain barrier, hybridization-triggered cleavage agents, or intercalating agents. An antisense molecule may comprise at least one modified base moiety, for example, 5-fluorouracil, 5-bromouracil, 5-chlorouracil, 5-iodouracil, hypoxanthine, xanthine, 4-acetylcytosine, 5-(carboxyhydroxytiethyl) uracil, etc. The molecule may also comprise at least one modified sugar moiety including arabinose, 2-fluoroarabinose, xylulose, and hexose. An antisense molecule can also contain a neutral peptide-like backbone, for example, peptide nucleic acid (PNA)-oligomers. Antisense oligonucleotides can be synthesized by standard methods known in the art, for example, by use of an automated DNA synthesizer (such as those commercially available from Biosearch, Applied Biosystems, etc).

“Binding agent” refers to a substance such as a polypeptide or antibody that specifically binds to one or more KD Marker. A substance “specifically binds” to one or more KD Marker if it reacts at a detectable level with one or more KD Marker, and does not react detectably with peptides containing an unrelated or different sequence. Binding properties may be assessed using an ELISA, which may be readily performed by those skilled in the art (see for example, Newton et al, Develop. Dynamics 197: 1-13, 1993). A binding agent may be a ribosome, with or without a peptide component, an aptamer, an RNA molecule, or a polypeptide. A binding agent may be a polypeptide that comprises one or more KD Marker sequence, a peptide variant thereof, or a non-peptide mimetic of such a sequence.

An aptamer includes a DNA or RNA molecule that binds to nucleic acids and proteins. An aptamer that binds to a protein (or binding domain) or a KD Polynucleotide can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

Antibodies for use in the present invention include but are not limited to monoclonal or polyclonal antibodies, immunologically active fragments (e.g. a Fab or (Fab)₂ fragments), antibody heavy chains, humanized antibodies, antibody light chains, genetically engineered single chain Fᵥ molecules (Ladner et al, U.S. Pat. No. 4,946,778), chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. Isolated native or recombinant KD Markers may be utilized to prepare antibodies. See, for example, Kohler et al. (1975) Nature 256:495-497; Kozbor et al. (1985) J. Immunol. Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies specific for a KD Marker may also be obtained from scientific or commercial sources. In an embodiment of the invention, antibodies are reactive against a KD Marker if they bind with a Kₐ of greater than or equal to 10⁻⁷ M.

A “chimeric polypeptide” of “fusion protein” comprises all or part (preferably biologically active) of a KD Polypeptide operably linked to a heterologous polypeptide (i.e., a polypeptide other than a KD Polypeptide). Within the fusion protein, the term “operably linked” is intended to indicate that a KD Polypeptide and the heterologous polypeptide are fused in-frame to each other. The heterologous polypeptide can be fused to the N-terminus or C-terminus of a KD Polypeptide. A useful fusion protein is a GST fusion protein in which a KD Marker is fused to the C-terminus of GST sequences. Another example of a fusion protein is an immunoglobulin fusion protein in which all or part of a KD Polypeptide is fused to sequences derived from a member of the immunoglobulin protein family. Chimeric and fusion proteins can be produced by standard recombinant DNA techniques.

A “fragment” or “portion” of a polypeptide may range in size from four amino acids to the entire amino acid minus one amino acid. A fragment or portion of a polypeptide can be a polypeptide which is for example, about 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide. A fragment can be an immunogenic portion of a KD Marker (e.g., an immunogenic portion of pVHL or CXCR4 Polypeptide or a pVHL or CXCR4 Interacting Polypeptide).

“Gene therapy” refers to the transfer and stable insertion of new genetic information into cells for the therapeutic treatment of conditions and/or diseases described herein. An exogenous gene (e.g., pVHL Polynucleotide) is transferred into a cell that proliferates to introduce the transferred gene throughout the cell population. Therefore, stem cells may be the target of gene transfer, since they will produce various lineages that will potentially express the exogenous gene. There are two approaches to gene therapy: (i) ex vivo or cellular gene therapy; and (ii) in vivo gene therapy. In ex vivo gene therapy cells are removed from a patient, and while being cultured are treated in vitro. An exogenous gene is introduced into the cells via an appropriate delivery vehicle/method (transfection, transduction, homologous recombination, etc.) and regulatory elements as required, and the modified cells are expanded in culture and returned to the patient. The genetically re-implanted cells express the transfected exogenous gene in situ. In in vivo gene therapy an exogenous gene is introduced into tissues and cells in subjects, for example, by systemic administration or direct injection into sites in situ. General references describing using stem cells as vehicles for gene therapy and clinical applications include Stem Cell Biology and Gene Therapy by P. J. Quesenberry et al., (eds), John Wiley & Sons, 1998; and Blood Cell Biochemistry: Hematopoiesis and Gene Therapy (Blood Cell Biochemistry, Vol. 8) by L. J. Fairbairn & N. G. Testa (eds)., Kluwer Academic Publishers, 1999.

“Host cells” include a wide variety of prokaryotic and eukaryotic host cells. For example, host cells include bacterial cells such as E. coli, Bacillus, or Streptomyces, insect cells (using baculovirus), yeast cells, or mammalian cells. Other suitable host cells can be found in Goeddel, Gene Expression Technology: Methods in Enzymology 185, Academic Press, San Diego, Calif. (1991). A host cell may also be chosen which modulates the expression of an inserted nucletotide sequence, or modifies (e.g. glycosylation or phosphorylation) and processes (e.g., cleaves) the polypeptide in a desired fashion. Host systems or cell lines may be selected which have specific and characteristic mechanisms for post-translational processing and modification of proteins. For long-term high-yield stable expression of the protein, cell lines and host systems which stably express the gene product may be engineered.

Host cells include stem cells i.e. cells that are capable under appropriate conditions of producing progeny of several different cell types that are derivatives of all of the three germinal layers (endoderm, mesoderm, and ectoderm). Stem cells include hematopoietic cells and may include stem cells of other origins such as stem cells from liver, pancreas, epithelium, neuron and bone marrow mesenchymal stem cells. Stem cells may be isolated from any known source of stem cells, and can be obtained from any tissue of any multicellular organism. The term includes cells obtained from primary tissue that are pluripotent and established cell lines of stem cells. Stem cells can also be derived from embryonic cells of various types, in particular, embryonic stem cells and more particularly initiated or differentiated embryonic stem cells.

“Identity” as known in the art and used herein, is a relationship between two or more amino acid sequences or two or more nucleic acid sequences, as determined by comparing the sequences. It also refers to the degree of sequence relatedness between amino acid or nucleic acid sequences, as the case may be, as determined by the match between strings of such sequences. Identity and similarity are well known terms to skilled artisans and they can be calculated by conventional methods (for example, see Computational Molecular Biology, Lesk, A. M. ed., Oxford University Press, New York, 1988; Biocomputing: Informatics and Genome Projects, Smith, D. W. ed., Academic Press, New York, 1993; Computer Analysis of Sequence Data, Part 1, Griffin, A. M. and Griffin, H. G. eds., Humana Press, New Jersey, 1994; Sequence Analysis in Molecular Biology, von Heinje, G. Academic Press, 1987; and Sequence Analysis Primer, Gribskov, M. and Devereux, J. eds. M. Stockton Press, New York, 1991, Carillo, H. and Lipman, D., SIAM J. Applied Math. 48:1073, 1988). Methods which are designed to give the largest match between the sequences are generally preferred. Methods to determine identity and similarity are codified in publicly available computer programs including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLASTX program is publicly available from NCBI and other sources (BLAST Manual, Altschul, S. et al. NCBI NLM NIH Bethesda, Md. 20894; Altschul, S. et al. J. Mol. Biol. 215: 403-410, 1990).

“Interacting Polypeptide” refers to a polypeptide that interacts with a KD Polypeptide, including a fragment thereof (e.g., domain). The terms “interact” and “interacting” refer to any physical association between molecules (e.g., polypeptides). The terms preferably refer to a stable association between two molecules due to, for example, electrostatic, hydrophobic, ionic and/or hydrogen-bond interactions under physiological conditions. Certain interacting or associated molecules interact only after one or more of them have been stimulated (e.g. phosphorylated). An interaction between proteins may be either direct or indirect.

An “isoform” refers to a polypeptide that contains the same number and kinds of amino acids as a KD Marker, but the isoform has a different molecular structure. The invention contemplates isoforms of a KD Marker or Interacting Polypeptide. Isoforms preferably have the same properties (e.g., biological and/or immunological activity) as a KD Marker or Interacting Polypeptide.

“Isolated” refers to polypeptides and polynucleotides removed from their natural environment, isolated or separated and that are at least 60%, 70%, 75%, 80%, 85%, 90%, 95%, 98%, or 99% free from other components which they are naturally associated. An isolated polynucleotide may also be free of sequences which naturally flank the nucleic acid (i.e., sequences located at the 5′ and 3′ ends of the polynucleotide) from which the nucleic acid is derived.

The term “KD Marker” includes a polypeptide marker associated with kidney disease. The term includes native-sequence polypeptides isoforms, chimeric polypeptides, complexes, fragments, precursors, modified forms, derivatives, any and all homologs and sequences with identity to same, and Interacting Polypeptides. The term in particular includes pVHL, VEGF-A, CXCR4, integrin β-1, PDGF-A, HIF1α, and TGFβ.

In an embodiment, a KD Marker is pVHL which includes the sequences of pVHL shown as SEQ ID NO. 1 or 2, Accession Nos. NP_{—}000542 and NP_{—}937799, or GeneID: 7428, or a fragment or isoform thereof.

In another embodiment, a KD Marker is VEGF-A which includes the sequences of VEGF-A shown as SEQ ID NO. 3, Accession Nos. NP_{—}001020537 to NP_{—}001020541, NP_{—}001028928, and NP_{—}003367, GeneID: 7422, or a fragment or isoform thereof.

In another embodiment, a KD Marker is CXCR4 which includes the sequences of CXCR4 shown as SEQ ID NO.4 and 5, Accession Nos. NP_{—}001008540 and NP_{—}003458, GeneID: 7852, or a fragment or isoform thereof.

In another embodiment, a KD Marker is HIF1α which includes the sequences of HIF1αshown as SEQ ID NO.6 and 7, Accession Nos. NP_{—}001521 and NP_{—}851397, GeneID: 3091, or a fragment or isoform thereof.

In another embodiment, a KD Marker is integrin β-1 which includes the sequences of integrin β-1 shown as SEQ ID NO. 8, Accession No. CAI14426, GeneID: 3688, or a fragment or isoform thereof.

In another particular embodiment, a KD Marker is TGFβ which includes the sequences of TGFβ shown as SEQ ID NO. 9, Accession No. NP_{—}000651, GeneID: 7040, or a fragment or isoform thereof.

In another embodiment, a KD Marker is PDGF-A which includes the sequences of PDGF-A shown as SEQ ID NOs. 10 and 11, Accession No. NP_{—}002598 and NP_{—}148983, GeneID: 5154, or a fragment or isoform thereof.

In some aspects the term “KD Marker” includes Interacting Polypeptides, in particular ligands of polypeptide markers associated with kidney disease, more particularly a ligand for CXCR4, most particularly stromal-derived factor-1 (SDF-1) [GeneID 6387; Accession Nos. CAC10202 and CAC10203; or SEQ ID NO. 28].

KD Markers may be prepared by recombinant or synthetic methods, or isolated from a variety of sources, or by any combination of these and similar techniques.

“KD Polynucleotides” refers to polynucleotides associated with kidney disease and/or encoding KD Markers including native-sequence polypeptides, polypeptide variants including a portion of a polypeptide, an isoform, precursor, complex, a chimeric polypeptide, or modified forms and derivatives of the polypeptides. KD Polynucleotides are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotides for use in the methods of the invention may be of any length suitable for a particular method. In certain applications the term refers to antisense polynucleotides (e.g. mRNA or DNA strand in the reverse orientation to sense polynucleotide markers).

A KD Polynucleotide includes polynucleotides encoding pVHL, VEGF-A, CXCR4, integrin β-1, PDGF-A, HIF1α and TGFβ.

In a particular embodiment, a KD Polynucleotide encodes pVHL which includes the nucleic acid sequences encoding pVHL shown in Accession Nos. NM_{—}000551, and NM_{—}198156, or GeneID: 7428, or a fragment thereof.

In another particular embodiment, a KD Polynucleotide encodes VEGF-A which includes the nucleic acid sequences encoding VEGF-A shown in Accession Nos. NM_{—}001025366, NP_{—}001020537, NP_{—}001020538, NM_{—}001025368. NP_{—}001020539, NM_{—}001025369, NM_{—}001025370, NM_{—}001033756, NP_{—}001028928, NM_{—}003376, or GeneID: 7422, or a fragment thereof.

In another particular embodiment, a KD Polynucleotide encodes CXCR4 which includes the nucleic acid sequences encoding CXCR4 shown in Accession Nos. NM_{—}001008540 and NM_{—}003467, or GeneID: 7852, or a fragment thereof.

In a particular embodiment, a KD Polynucleotide encodes HIF1α which includes the sequences encoding HIF1α, shown in Accession No. NM_{—}001530 and NM_{—}181054, or GeneID: 3091, or a fragment thereof.

In another particular embodiment, a KD Polynucleotide encodes integrin β-1 which includes the sequences of integrin β-1 shown in Accession Nos. NM_{—}002211, NM_{—}033666, NM_{—}033667, NM_{—}033668NM_{—}033669, NM_{—}133376, AL365203, U33879, U33880, U333882, and X68969, GeneID: 3688, or a fragment thereof.

In another particular embodiment, a KD Polynucleotide encodes TGFβ1 which includes the sequences of TGFβ1 shown in Accession No. NM_{—}000660, AY059373, AY330201, AY330202, and GeneID: 7040, or a fragment thereof.

In another particular embodiment, a KD Polynucleotide encodes PDGF-A which includes the sequences of PDGF-A shown in Accession Nos. NM_{—}002607 and NM_{—}033023, or GeneID: 5154, or a fragment thereof.

KD Polynucleotides include complementary nucleic acid sequences, and nucleic acids that are substantially identical to these sequences (e.g. at least about 45%, preferably 50%, 55%, 60%, 65%, 70%, 75%, 80%, 85%, 90%, 95%, 97%, 98%, or 99% identity).

KD Polynucleotides also include sequences that differ from a native sequence due to degeneracy in the genetic code. As one example, DNA sequence polymorphisms within the nucleotide sequence of a KD Polynucleotide may result in silent mutations that do not affect the amino acid sequence. Variations in one or more nucleotides may exist among individuals within a population due to natural allelic variation. DNA sequence polymorphisms may also occur which lead to changes in the amino acid sequence of a polypeptide. KD Polynucleotides also include nucleic acids that hybridize under stringent conditions, preferably high stringency conditions to a KD Polynucleotide.

KD Polynucleotides also include truncated nucleic acids or nucleic acid fragments and variant forms of the nucleic acids that arise by alternative splicing of an mRNA corresponding to a DNA.

“Kidney disease”, refers to kidney disorders or glomerular diseases include without limitation glomerulonephritis (i.e., inflammation of the membrane tissue in the kidney that serves as a filter, separating wastes and extra fluid from the blood) and glomerulosclerosis (scarring or hardening of the tiny blood vessels within the kidney) A number of different diseases can result in a glomerular disease. It may be the direct result of an infection or a drug toxic to the kidneys, or it may result from a disease that affects the entire body, like diabetes or lupus. Many different kinds of diseases can cause swelling or scarring of the glomerulus. Sometimes glomerular disease is idiopathic i.e., occurs without an apparent associated disease. Examples of diseases that can cause glomerular disease include without limitation autoimmune diseases such as systemic lupus erythematosus, Goodpasture's syndrome, and IgA nephropathy; hereditary nephritis such as Alport Syndrome; infection-related glomoerular disease including acute post-streptococcal glomerulonephritis, bacterial endocarditis, and HIV-associated nephropathy; and, sclerotic diseases such as glomerulosclerosis, diabetic nephropathy, and focal segmental glomerulosclerosis. Other examples of glomerular diseases included membranous nephropathy and minimal change disease.

A kidney disease particularly includes renal glomerulonephritis, in particular rapid progressive glomerulonephritis, renal fibrosis, or both. These conditions can be associated with, for example, Alport syndrome, IDDM nephritis, mesangial proliferative glomerulonephritis, membrano proliferative glomerulonephritis, crescentic glomerulonephritis, diabetic nephropathy, and renal insterstitial fibrosis.

In certain aspects of the invention, a kidney disease is RPGN, most particularly pauci-immune RPGN.

In certain other aspects, a kidney disease is IgA nephropathy.

“Micro-array” and “array” refer to nucleic acid or nucleotide arrays or protein or peptide arrays that can be used to detect biomolecules associated with kidney disease, for instance to measure gene expression. A variety of arrays are made in research and manufacturing facilities worldwide, some of which are available commercially. By way of example, spotted arrays and in situ synthesized arrays are two kinds of nucleic acid arrays that differ in the manner in which the nucleic acid materials are placed onto the array substrate. A widely used in situ synthesized oligonucleotide array is GeneChip™ made by Affymetrix, Inc. Oligonucleotide probes that are 20- or 25-base long can be synthesized in silico on the array substrate. These arrays can achieve high densities (e.g., more than 40,000 genes per cm²). Generally spotted arrays have lower densities, but the probes, typically partial cDNA molecules, are much longer than 20- or 25-mers. Examples of spotted cDNA arrays include LifeArray made by Incyte Genomics and DermArray made by IntegriDerm (or Invitrogen). Pre-synthesized and amplified cDNA sequences are attached to the substrate of spotted arrays. Protein and peptide arrays also are known [(see for example, Zhu et al., Science 293:2101 (2001)].

“Mimetic” refers to a synthetic chemical compound that has substantially the same structural and/or functional characteristics of a KD Marker. A mimetic can be composed entirely of synthetic, non-natural analogues of amino acids, or, is a chimeric molecule of partly natural peptide amino acids and partly non-natural analogs of amino acids. A polypeptide can be characterized as a mimetic when all or some of its residues are joined by chemical means other than natural peptide bonds (see, e.g., Spatola (1983) in Chemistry and Biochemistry of Amino Acids, Peptides and Proteins, Vol. 7, pp 267-357, “Peptide Backbone Modifications,” Marcell Dekker, N.Y.). Mimetics also include peptoids, oligopeptoids (Simon et al (1972) Proc. Natl. Acad, Sci USA 89:9367); and peptide libraries containing peptides of a designed length representing all possible sequences of amino acids corresponding to a motif or peptide. A particular mimetic refers to a molecule, the structure of which is developed based on the structure of a KD Marker or portions thereof, and is able to effect some of the actions of chemically or structurally related molecules.

“Modified forms” of a KD Marker includes modified forms of the polypeptides and derivatives of the polypeptides, including but not limited to glycosylated, phosphorylated, acetylated, methylated or lapidated forms of the polypeptides.

“Modulate” refers to a change or an alteration in the activity of a KD Marker, in particular the biological and/or immunological activity of a KD Marker. Modulation may be an increase or decrease in the activity of a KD Marker, a change in binding characteristics, or any other changes in the biological, functional, or immunological properties of a KD Marker. “Biological activity” refers to structural, regulatory, or biochemical functions of a naturally occurring molecule. “Immunological activity” refers to induction of an immune response by a natural, synthetic or recombinant KD Marker, or any fragment thereof, in appropriate subjects or cells, and/or binding of a natural, synthetic or recombinant KD Marker with specific antibodies.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers etc. include but are not limited to saline, buffered saline, dextrose, water, glycerol, ethanol, and combinations thereof. The use of such media and agents for an active substance is well known in the art.

A “polypeptide analogue” includes a polypeptide wherein one or more amino acid residues of a native polypeptide have been substituted by another amino acid residue, one or more amino acid residues of a native polypeptide have been inverted, one or more amino acid residues of the native polypeptide have been deleted, and/or one or more amino acid residues have been added to the native polypeptide. Such an addition, substitution, deletion, and/or inversion may be at either of the N-terminal or C-terminal end or within the native polypeptide, or a combination thereof.

A “polypeptide derivative” includes a polypeptide in which one or more of the amino acid residues of a native polypeptide have been chemically modified. A chemical modification includes adding chemical moieties, creating new bonds, and removing chemical moieties. A polypeptide may be chemically modified, for example, by alkylation, acylation, glycosylation, pegylation, ester formation, deamidation, or amide formation.

A “polypeptide variant” refers to a polypeptide having at least about 60%, 65%, 70%, 75%, 80%, 85%, 90%, 95%, 97%, 98%, or 99% amino acid sequence identity, particularly at least about 70-80%, more particularly at least about 85%, still more particularly at least about 90%, most particularly at least about 95% amino acid sequence identity with a native-sequence polypeptide. Particular KD Marker variants have at least 85%, 90%, 95% amino acid sequence identity to the sequences identified in SEQ ID NOs. 1 to 11. Such variants include for instance polypeptides wherein one or more amino acid residues are added to, or deleted from the N- or C-terminus of the full-length or mature sequences of the polypeptide, including variants from other species, but excludes a native-sequence polypeptide. In aspects of the invention variants retain the immunogenic activity of the corresponding native-sequence polypeptide. A naturally occurring allelic variant may contain conservative amino acid substitutions from the native polypeptide sequence or it may contain a substitution of an amino acid from a corresponding position in a polypeptide homolog, for example, a murine polypeptide.

Mutations may be introduced into a polypeptide by standard methods, such as site-directed mutagenesis and PCR-mediated mutagenesis. Conservative substitutions can be made at one or more predicted non-essential amino acid residues. A conservative amino acid substitution is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Computer programs, for example DNASTAR, may be used to determine which amino acid residues may be substituted, inserted, or deleted without abolishing biological and/or immunological activity. Following mutagenesis the variant polypeptide can be recombinantly expressed.

A “probe” to which a particular KD Polynucleotide molecule specifically hybridizes contains a complementary genomic polynucleotide sequence. The nucleotide sequences of the probes can be about 10-200 nucleotides in length. The probes can be genomic sequences of a species of organism, such that a plurality of different probes is present, with complementary sequences capable of hybridizing to the genome of such a species of organism. In aspects of the invention, the probes are about 10-30, 10-40, 20-50, 40-80, 50-150, 80-120 nucleotides in length, and in particular about 60 nucleotides in length.

The probes may comprise DNA or DNA mimics (e.g., derivatives and analogues) corresponding to a portion of an organism's genome, or complementary RNA or RNA mimics. Mimics are polymers comprising subunits capable of specific, Watson-Crick-like hybridization with DNA, or of specific hybridization with RNA. The nucleic acids can be modified at the base moiety, at the sugar moiety, or at the phosphate backbone.

DNA can be obtained using standard methods such as polymerase chain reaction (PCR) amplification of genomic DNA or cloned sequences. (See, for example, in Innis et al., eds., 1990, PCR Protocols: A Guide to Methods and Applications, Academic Press Inc., San Diego, Calif.). Computer programs known in the art can be used to design primers with the required specificity and optimal amplification properties, such as Oligo version 5.0 (National Biosciences). Controlled robotic systems may be useful for isolating and amplifying nucleic acids.

Probes for a microarray can be synthesized using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 10 and about 500 bases, 20-100 bases, or 40-70 bases in length. Synthetic nucleic acid probes can include non-natural bases, such as, without limitation, inosine. Nucleic acid analogues such as peptide nucleic acid may be used as binding sites for hybridization. (see, e.g., Egholm et al., 1993, Nature 363:566-568; U.S. Pat. No. 5,539,083).

Probes can be selected using an algorithm that takes into account binding energies, base composition, sequence complexity, cross-hybridization binding energies, and secondary structure (see Friend et al., International Patent Publication WO 01/05935, published Jan. 25, 2001).

Positive control probes, (e.g., probes known to be complementary and hybridizae to sequences in the target polynucleotides), and negative control probes, (e.g., probes known to not be complementary and hybridize to sequences in the target polynucleotides) are typically included on the array. Positive controls can be synthesized along the perimeter of the array or synthesized in diagonal stripes across the array. A reverse complement for each probe can be next to the position of the probe to serve as a negative control.

The probes can be attached to a solid support or surface, which may be made from glass, plastic (e.g., polypropylene, nylon), polyacrylamide, nitrocellulose, gel, or other porous or nonporous material. The probes can be printed on surfaces such as glass plates (see Schena et al., 1995, Science 270:467-470). This method may be particularly useful for preparing microarrays of cDNA (See also, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

“Regulatory element” refers to a genetic element or elements having a regulatory role in gene expression, for example, promoters or enhancers. Large numbers of suitable vectors and promoters are known to those of skill in the art and are commercially available for generating recombinant constructs encoding a KD Marker or chimeric polypeptide. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTL pSG (Stratagene) pSVK3, pBPV, pMSG, pSVL (Pharmacia). As defined herein “operably linked” means that an isolated polynucleotide and a regulatory element are situated within a vector or cell in such a way that the polypeptide is expressed by a host cell which has been transformed (transfected) with the ligated polynucleotide/regulatory element sequence. A regulatory element can be a constitutive or induced transcriptional regulatory region, for example, a transcriptional regulatory region from an insulin gene that is induced by increasing intracellular glucose concentrations.

The term “sample” is used in its broadest sense. Samples that may be analyzed using the methods of the invention include those which are known or suspected to express or contain a KD Polypeptide, KD Polynucleotide, Interacting Polypeptide, protein complexes, or antibodies specific for a KD Polypeptide. A sample can be used directly as obtained from the source or following a pretreatment to modify the character of the sample. The sample can be derived from any biological source, such as tissues, extracts, or cell cultures, including cells, cell lysates, and physiological fluids, such as, for example, whole blood, plasma, serum, saliva, lymph fluid, follicular fluid, seminal fluid, amniotic fluid, sputum, tears, perspiration, mucus, ocular lens fluid, cerebral spinal fluid, sweat, urine, milk, ascites fluid, synovial fluid, peritoneal fluid and the like. The sample can be treated prior to use, such as preparing plasma from blood, diluting viscous fluids, and the like. Methods of treatment can involve filtration, distillation, extraction, concentration, inactivation of interfering components, the addition of reagents, and the like. Proteins may be isolated from the samples and utilized in the methods of the invention.

In embodiments of the invention the sample is blood.

The samples that may be analyzed in accordance with the invention include polynucleotides from clinically relevant sources, preferably expressed RNA or a nucleic acid derived therefrom (cDNA or amplified RNA derived from cDNA that incorporates an RNA polymerase promoter). The target polynucleotides can comprise RNA, including, without limitation total cellular RNA, poly(A)⁺ messenger RNA (mRNA) or fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. No. 5,545,522, 5,891,636, or 5,716,785). Methods for preparing total and poly(A)⁺ RNA are well known in the art, and are described generally, for example, in Sambrook et al., (1989, Molecular Cloning—A Laboratory Manual (2^{nd} Ed.), Vols. 1-3, Cold Spring Harbor Laboratory, Cold Spring Harbor, N.Y.) and Ausubel et al, eds. (1994, Current Protocols in Moelcular Biology, vol. 2, Current Protocols Publishing, New York). RNA may be isolated from eukaryotic cells by procedures involving lysis of the cells and denaturation of the proteins contained in the cells. Additional steps may be utilized to remove DNA. Cell lysis may be achieved with a nonionic detergent, followed by microcentrifugation to remove the nuclei and hence the bulk of the cellular DNA. (See Chirgwin et al., 1979, Biochemistry 18:5294-5299). Poly(A)+RNA can be selected using oligo-dT cellulose (see Sambrook et al., 1989, Molecular Cloning—A Laboratory Manual (2nd Ed.), Vols. 1-3, Cold Spring Harbor Laboratory, Cold Spring Harbor, N.Y.). In the alternative, RNA can be separated from DNA by organic extraction, for example, with hot phenol or phenol/chloroform/isoamyl alcohol.

It may be desirable to enrich mRNA with respect to other cellular RNAs, such as transfer RNA (tRNA) and ribosomal RNA (rRNA). Most mRNAs contain a poly(A) tail at their 3′ end allowing them to be enriched by affinity chromatography, for example, using oligo(dT) or poly(U) coupled to a solid support, such as cellulose or Sephadex™ (see Ausubel et al., eds., 1994, Current Protocols in Molecular Biology, vol. 2, Current Protocols Publishing, New York). Bound poly(A)+mRNA is eluted from the affinity column using 2 mM EDTA/0.1% SDS.

A sample of RNA can comprise a plurality of different mRNA molecules each with a different nucleotide sequence. In an aspect of the invention, the mRNA molecules in the RNA sample comprise at least 100 different nucleotide sequences.

Target polynucleotides can be detectably labeled at one or more nucleotides using methods known in the art. The label is preferably uniformly incorporated along the length of the RNA, and more preferably, is carried out at a high degree of efficiency. The detectable label can be a luminescent label, fluorescent label, bio-luminescent label, chemi-luminescent label, radiolabel, and colorimetric label. In a particular embodiment, the label is a fluorescent label, such as a fluorescein, a phosphor, a rhodamine, or a polymethine dye derivative. Commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.).

Target polynucleotides from a patient sample can be labeled differentially from polynucleotides of a standard. The standard can comprise target polynucleotides from normal individuals (i.e., those not afflicted with or pre-disposed to kidney disease), in particular pooled from samples from normal individuals. The target polynucleotides can be derived from the same individual, but taken at different time points, and thus indicate the efficacy of a treatment by a change in expression of the markers, or lack thereof, during and after the course of treatment.

“Short interfering RNA” or “siRNA” refer to short interfering RNA polynucleotides that are capable of sequence-specific post transcriptional gene silencing. In particular the term refers to a double stranded nucleic acid molecule capable of RNA interference (RNAi). (See for example Bass, 2001, Nature, 411, 428-429; Elbashir et al., 2001, Nature, 411, 494-498; and Kreutzer et al., PCT Publication No. WO 00/44895; Zernicka-Goetz et al., PCT Publication No. WO 01/36646; Fire, PCT Publication No. WO 99/32619; Plaetinck et al., PCT Publication No. WO 00/01846; Mello and Fire, PCT Publication No. WO 01/29058; Deschamps-Depaillette, PCT Publication No. WO 99/07409; and Li et al., PCT Publication No. WO 00/44914. An siRNA molecule can have a length from about 10-50 or more nucleotides (or nucleotide analogs), about 15-25 nucleotides (or nucleotide analogs), or about 20-23 nucleotides (or nucleotide analogs). As used herein, siRNA molecules are not limited to those molecules containing only RNA, but further encompasses chemically modified nucleotides and non-nucleotides

“Statistically different levels”, “significantly altered”, or “significant difference” in levels of markers in a patient sample compared to a control or standard (e.g. normal levels or levels in other samples from a patient) may represent levels that are higher or lower than the standard error of the detection assay. In particular embodiments, the levels may be at least about 1.5, 2, 3, 4, 5, or 6 times higher or lower than the control or standard.

“Stringent conditions” as used herein refers to low or high stringency conditions. Factors including the length and nature of the sequence, nature of the target (DNA, RNA, base composition), and the concentration of the salts and other components (for example, the presence or absence of formamide, dextran sulfate, and/or polyethylene glycol) and the hybridization solution can be varied to generate conditions of either low or high stringency.

In aspects of the invention the term refers to the stringency that occurs within a range from about Tm −5° C. (5° C. below the melting temperature (Tm) of the probe) to about 20° C. to 25° C. below Tm. Stringency conditions may be altered in order to identify or detect identical or related polynucleotide sequences. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

The terms “subject”, “individual”, “recipient” or “patient” refer to an animal including a warm-blooded animal such as a mammal, which is afflicted with or suspected of having or being pre-disposed to a kidney disease. Mammal includes without limitation any members of the Mammalia. In general, the terms refer to a human. The terms also include domestic animals bred for food or as pets, including horses, cows, sheep, poultry, fish, pigs, cats, dogs, and zoo animals, goats, apes (e.g. gorilla or chimpanzee), and rodents such as rats and mice. The methods herein for use on subjects/individuals/patients contemplate prophylactic as well as curative use.

Typical subjects contemplated by the invention include persons susceptible to, suffering from or that have suffered kidney disease.

“Transformant host cells” include host cells which have been transformed or transfected with a recombinant expression vector of the invention. The terms “transformed with”, “transfected with”, “transformation” and “transfection” encompass the introduction of a nucleic acid (e.g., a vector) into a host cell by one of many standard techniques. Prokaryotic cells can be transformed with a polynucleotide by, for example, electroporation or calcium-chloride mediated transformation. A polynucleotide can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

The term “treating” refers to reversing, alleviating, or inhibiting the progress of a disease, or one or more symptoms of such disease, to which such term applies. Depending on the condition of the subject, the term also refers to preventing a disease, and includes preventing the onset, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a therapeutic to a subject that is not at the time of administration afflicted with the disease. “Preventing” also refers to preventing the recurrence of a disease or of one or more symptoms associated with such disease. The terms “treatment” and “therapeutically,” refer to the act of treating, as “treating” is defined above.

Identification of Markers

The present invention provides sets of markers for detecting, diagnosing and predicting kidney disease or onset of kidney disease in patient samples. Generally, marker sets can be identified by determining which human markers have expression patterns that correlated with kidney disease.

Thus, the invention relates to a method of characterizing a sample by detecting or quantitating in the sample one or more polypeptides or polynucleotides extracted from the sample that are characteristic of kidney disease the method comprising assaying for differential expression of polypeptides or polynucleotides in the sample. Differential expression of the polynucleotides can be determined by micro-array analysis or by amplification of the extracted polynucleotides.

In an embodiment, a method for identifying sets or markers is provided comprising extracting and labeling target polypeptides or polynucleotides, and comparing the expression of all markers (polypeptides or genes) in a sample to the expression of all markers in a standard or control. The sample may comprise a single sample, or a pool of samples; the samples in the pool may come from different individuals. In one embodiment, the standard or control comprises target polypeptides or polynucleotides derived from a sample from a normal individual (i.e., an individual not afflicted or pre-disposed to kidney disease). In a particular embodiment, the standard or control is a pool of target polypeptides or polynucleotides derived from collected samples from a number of normal individuals.

Comparison of the patient sample and control may be accomplished by any means known in the art. By way of example, expression levels of various markers can be assessed by separation of target polynucleotides (e.g., RNA or cDNA) derived from the markers in agarose or polyacrylamide gels, followed by hybridization with marker-specific oligonucleotide probes. In the alternative, the comparison may be accomplished by the labeling of target polynucleotides followed by separation on a sequencing gel. The patient and control or standard polynucleotides can be in adjacent lanes. Expression levels can be compared visually or using a densitometer. In a particular embodiment, the expression of all markers is assessed simultaneously by hybridization to an oligonucleotide microarray. In each approach, markers meeting certain criteria are identified as associated with kidney disease.

Markers can be selected based upon a significant difference of expression (up- or down-regulation) in a sample as compared to a standard or control. Markers can also be selected by calculation of the statistical significance (i.e., the p-value) of the correlation between the expression of the marker and kidney disease. Both selection criteria are generally used. In an aspect of the invention, markers associated with kidney disease are selected where the markers show more than two-fold change (increase or decrease) in expression as compared to a standard, and/or the p-value for the correlation between kidney disease and the change in marker expression is no more than 0.01 (i.e., is statistically significant).

The expression of the identified kidney disease markers can be used to identify markers that can differentiate kidney disease into clinical types.

A profile of nucleic acids can be produced by a microarray or by amplification of the nucleic acids (e.g. using PCR).

In an aspect the invention provides a method of characterizing a sample by detecting or quantitating in the sample one or more polynucleotides extracted from the sample that are characteristic of kidney disease the method comprising assaying for differential expression of polynucleotides in the sample by microarray of polynucleotides extracted from the sample.

The invention also relates to a method of characterizing a sample by detecting or quantitating in the sample one or more polypeptides extracted from the sample that are characteristic of kidney disease the method comprising assaying for differential expression of polypeptides in the sample. Differential expression of polypeptides can be assayed by mass spectroscopy or an antibody microarray of polypeptides extracted from the sample.

Therefore, the invention relates to a method for identifying KD Polypeptides associated with kidney disease comprising:
(a) obtaining a sample from a subject;
(b) extracting polypeptides from the sample and producing a profile of the polypeptides by subjecting the polypeptides to mass spectrometry; and
(c) comparing the profile with a profile for a normal sample or for a known stage or type of kidney disease to identify polypeptides associated with kidney disease.

Polypeptides may be extracted from the samples in a manner known in the art. For example, polypeptides may be extracted by first digesting or disrupting cell membranes by standard methods such as detergents or homogenization in an isotonic sucrose solution, followed by ultra-centrifugation or other standard techniques.

The separated polypeptides may be digested into peptides, in particular using proteolytic enzymes such as trypsin, pepsin, subtilisin, and proteinase. For example, polypeptides may be treated with trypsin which cleaves at the sites of lysine and arginine, to provide doubly-charged peptides with a length of from about 5 to 50 amino acids. Such peptides may be particularly appropriate for mass spectrometry analysis, especially electrospray ionization mass spectrometry. Chemical reagents including cyanogen bromide may also be utilized to digest proteins.

Mass spectrometers that may be used to analyze the peptides or polypeptides include a Matrix-Assisted Laser Desorptioon/loniation Time-of-Flight Mass Spectrometer (“MALDI-TOF”) (e.g. from PerSeptive Biosystems, Framingham, Mass.); an Electrospray Ionization (“ESI”) ion trap spectrometer, (e.g. from Finnigan MAT, San Jose, Calif.), an ESI quadrupole mass spectrometer (e.g. from Finnigan or Perkin-Elmer Corporation, Foster City, Calif.), a quadrupole/TOF hybrid tandem mass spectrometer, QSTAR XL (Applied Biosystems/MDS Sciex), or a Surface Enhanced Laser Desorption/Ionization (SELDI-TOF) Mass Spectrometer (e.g. from Ciphergen Biosystems Inc.).

Detection Methods

A variety of methods can be employed for the detection, diagnosis, monitoring, and prognosis of kidney disease, onset of kidney disease, or status of kidney disease involving one or more KD Markers and/or KD Polynucleotides, and for the identification of subjects with a predisposition to kidney disease. Such methods may, for example, utilize KD Polynucleotides, and fragments thereof, and binding agents (e.g. antibodies) against one or more KD Markers, including peptide fragments. In particular, the polynucleotides and antibodies may be used, for example, for (1) the detection of the presence of KD Polynucleotide mutations, or the detection of either an over- or under-expression of KD Polynucleotide mRNA relative to a non-pre-term state, or the qualitative or quantitative detection of alternatively spliced forms of KD Polynucleotide transcripts which may correlate with certain conditions or susceptibility toward kidney disease; and (2) the detection of either an over- or an under-abundance of one or more KD Markers relative to a non-kidney disease state or a different stage or type of injury or the presence of a modified (e.g., less than full length) KD Marker which correlates with a kidney disease state or a progression toward kidney disease, or a particular type or stage of kidney disease.

If the gene(s) represent surface antigens or secreted peptides, antibodies can be raised and standard ELISA's developed. In addition, novel automated RNA extraction can be utilized, followed by multiplex, real time RT-PCR. For example, the MagNA Pure LC & LightCycler system from Roche Diagnostic is capable of accurately quantifying RNA expression in cells within 90 minutes.

The invention contemplates a method for detecting or monitoring the stage or type of kidney disease or onset of kidney disease, comprising producing a profile of levels of one or more KD Markers and/or KD Polynucleotides, and optionally other markers associated with kidney disease in a sample from a patient, and comparing the profile with a reference to identify a profile for the patient indicative of the stage or type of kidney disease.

The methods described herein may be used to evaluate the probability of the presence of kidney disease or onset of kidney disease, for example, in a sample freshly removed from a host. Such methods can be used to detect kidney disease and help in the diagnosis and prognosis of kidney disease. The methods can be used to detect the potential for kidney disease and to monitor kidney disease or a therapy.

The invention also contemplates a method for detecting kidney disease or onset of kidney disease comprising producing a profile of levels of one or more KD Markers and/or KD Polynucleotides, and other markers associated with kidney disease in a sample (e.g. cells) from a patient, and comparing the profile with a reference to identify a profile for the patient indicative of kidney disease.

The methods described herein can be adapted for diagnosing and monitoring kidney disease by detecting one or more KD Markers or KD Polynucleotides in biological samples from a subject. These applications require that the amount of KD Markers or KD Polynucleotides quantitated in a sample from a subject being tested be compared to a predetermined standard or cut-off value. The standard may correspond to levels quantitated for another sample or an earlier sample from the subject, or levels quantitated for a control sample. Levels for control samples from healthy subjects, different stages or types of kidney disease, may be established by prospective and/or retrospective statistical studies. Healthy subjects who have no clinically evident kidney disease or abnormalities may be selected for statistical studies. Diagnosis may be made by a finding of statistically different levels of detected KD Markers associated with kidney disease or KD Polynucleotides, compared to a control sample or previous levels quantitated for the same subject.

The methods described herein may also use multiple markers for kidney disease. Therefore, the invention contemplates a method for analyzing a biological sample for the presence of one or more KD Markers and KD Polynucleotides and other markers that are specific indicators of kidney disease. The methods described herein may be modified by including reagents to detect the additional markers.

Nucleic Acid Methods/Assays

As noted herein kidney disease or stage or type of same may be detected based on the level of KD Polynucleotides in a sample. Techniques for detecting polynucleotides such as polymerase chain reaction (PCR) and hybridization assays are well known in the art.

Probes may be used in hybridization techniques to detect polynucleotide markers. The technique generally involves contacting and incubating polynucleotides (e.g. recombinant DNA molecules, cloned genes) obtained from a sample from a patient or other cellular source with a probe under conditions favourable for the specific annealing of the probes to complementary sequences in the polynucleotides. After incubation, the non-annealed nucleic acids are removed, and the presence of polynucleotides that have hybridized to the probe if any are detected.

Nucleotide probes for use in the detection of nucleic acid sequences in samples may be constructed using conventional methods known in the art. Suitable probes may be based on nucleic acid sequences encoding at least 5 sequential amino acids from regions of a KD Polynucleotide, preferably they comprise 10-30, 10-40, 15-40, 20-50, 40-80, 50-150, or 80-120 nucleotides.

A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect KD Polynucleotides in human samples, e.g. blood or serum. The nucleotide probes may also be useful in the diagnosis of kidney disease involving one or more KD Polynucleotides; in monitoring the progression of kidney disease; or monitoring a therapeutic treatment.

The levels of mRNA or polynucleotides derived therefrom can be determined using hybridization methods known in the art. For example, RNA can be isolated from a sample and separated on a gel. The separated RNA can then be transferred to a solid support and nucleic acid probes representing one or more markers can be hybridized to the solid support and the amount of marker-derived RNA is determined. Such determination can be visual, or machine-aided (e.g. use of a densitometer). Dot-blot or slot-blot may also be used to determine RNA. RNA or nucleic acids derived therefrom from a sample are labeled, and then hybridized to a solid support containing oligonucleotides derived from one or more marker genes that are placed on the solid support at discrete, easily-identifiable locations. Hybridization, or the lack thereof, of the labeled RNA to the solid support oligonucleotides is determined visually or by densitometer.

The detection of KD Polynucleotides may involve the amplification of specific gene sequences using an amplification method such as polymerase chain reaction (PCR), followed by the analysis of the amplified molecules using techniques known to those skilled in the art. Suitable primers can be routinely designed by one of skill in the art.

By way of example, at least two oligonucleotide primers may be employed in a PCR based assay to amplify a portion of a KD Polynucleotide(s) derived from a sample, wherein at least one of the oligonucleotide primers is specific for (i.e. hybridizes to) a KD Polynucleotide. The amplified cDNA is then separated and detected using techniques well known in the art, such as gel electrophoresis.

In order to maximize hybridization under assay conditions, primers and probes employed in the methods of the invention generally have at least about 60%, preferably at least about 75%, and more preferably at least about 90% identity to a portion of a KD Polynucleotide; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of KD Polynucleotide expression. For example, RNA may be isolated from a cell type or tissue known to express a KD Polynucleotide and tested utilizing the hybridization (e.g. standard Northern analyses) or PCR techniques referred to herein. The primers and probes may be used in the above-described methods in situ i.e. directly on tissue sections (fixed and/or frozen) of patient tissue obtained from biopsies or resections.

In an aspect of the invention, a method is provided employing reverse transcriptase-polymerase chain reaction (RT-PCR), in which PCR is applied in combination with reverse transcription. Generally, RNA is extracted from a sample using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against a KD Polynucleotide sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with a suspected kidney disease and an individual who is not predisposed to kidney disease. The reaction may be performed on several dilutions of cDNA spanning at least two orders of magnitude. A significant difference in expression in several dilutions of the subject sample as compared to the same dilutions of the normal sample may be considered positive for the presence of kidney disease.

In an embodiment, the invention provides methods for determining the presence or absence of a kidney disease in a subject comprising (a) contacting a sample obtained from the subject with oligonucleotides that hybridize to one or more KD Polynucleotides; and (b) detecting in the sample a level of nucleic acids that hybridize to the polynucleotides relative to a predetermined cut-off value, and therefrom determining the presence or absence of kidney disease in the subject.

The invention provides a method wherein a KD Polynucleotide which is mRNA is detected by (a) isolating mRNA from a sample and combining the mRNA with reagents to convert it to cDNA; (b) treating the converted cDNA with amplification reaction reagents and nucleic acid primers that hybridize to one or more KD Polynucleotides to produce amplification products; (d) analyzing the amplification products to detect amounts of mRNA encoding KD Polynucleotides; and (e) comparing the amount of mRNA to an amount detected against a panel of expected values for normal tissue derived using similar nucleic acid primers.

KD Polypeptides-positive samples or alternatively higher levels in patients compared to a control (e.g. normal tissue) may be indicative of kidney disease or advanced kidney disease, and/or that the patient is not responsive to or tolerant of a therapy. Alternatively, negative samples or lower levels compared to a control (e.g. normal samples or negative samples) may also be indicative of kidney disease or advanced kidney disease.

In another embodiment, the invention provides methods for determining the presence or absence of kidney disease in a subject comprising (a) contacting a sample obtained from the subject with oligonucleotides that hybridize to one or more KD Polynucleotides; and (b) detecting in the sample levels of polynucleotides that hybridize to the KD Polynucleotides relative to a predetermined cut-off value, and therefrom determining the presence or absence of kidney disease in the subject. In an embodiment, the KD Polynucleotides encode one or more of pVHL, VEGF-A, CXCR4, integrin β-1, PDGF-A, HIF1α, and TGFβ.

In a particular aspect, the invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to a KD Polynucleotide, to produce amplification products; (d) analyzing the amplification products to detect an amount of KD Polynucleotide mRNA; and (e) comparing the amount of mRNA to an amount detected against a panel of expected values for normal subjects derived using similar nucleic acid primers.

In another particular aspect, the invention provides a method wherein KD Polynucleotides that are mRNA are detected by (a) isolating mRNA from a sample and combining the mRNA with reagents to convert it to cDNA; (b) treating the converted cDNA with amplification reaction reagents and nucleic acid primers that hybridize to a KD Polynucleotide, to produce amplification products; (d) analyzing the amplification products to detect an amount of KD Polynucleotide mRNA; and (e) comparing the amount of mRNA to an amount detected against a panel of expected values for normal subjects derived using similar nucleic acid primers.

Marker-positive samples or alternatively higher levels, in particular significantly higher levels of KD Polynucleotides encoding one or more of VEGF-A, CXCR4, integrin β-1, PDGF-A, HIF1α and TGFβ in patients compared to a control (e.g. normal) are indicative of kidney disease, in particular RPGN, more particularly pauci-immune RPGN.

Marker-negative samples or alternatively lower levels, in particular significantly lower levels of KD Polynucleotides encoding pVHL in patients compared to a control (e.g. normal) are indicative of kidney disease, in particular RPGN, more particularly pauci-immune RPGN.

Marker-negative samples or alternatively lower levels, in particular significantly lower levels of KD Polynucleotides encoding one or more of VEGF-A, CXCR4, integrin β-1, PDGF-A, HIF1α and TGFβ in patients compared to a control (e.g. normal) are indicative of kidney disease, in particular IgA nephropathy.

Oligonucleotides or longer fragments derived from KD Polynucleotides may be used as targets in a micro-array as described herein. The micro-array can be used to simultaneously monitor the expression levels of large numbers of genes. The micro-array can also be used to identify genetic variants, mutations, and polymorphisms. The information from the micro-array may be used to determine gene function, to understand the genetic basis of kidney disease, to diagnose kidney disease, and to develop and monitor the activities of therapeutic agents.

Thus, the invention also includes an array comprising one or more KD Polynucleotidess (in particular pVHL, VEGF-A, CXCR4, integrin β-1, PDGF-A, HIF1α, HIF2α, and TGFβ), and optionally other markers. The array can be used to assay expression of KD Polynucleotides in the array. The invention allows the quantitation of expression of one or more KD Polynucleotides.

Micro-arrays typically contain at separate sites nanomolar quantities of individual genes, cDNAs, or ESTs on a substrate (e.g. nitrocellulose or silicon plate), or photolithographically prepared glass substrate. The arrays are hybridized to cDNA probes using conventional techniques with gene-specific primer mixes. The target polynucleotides to be analyzed are isolated, amplified and labeled, typically with fluorescent labels, radiolabels or phosphorous label probes. After hybridization is completed, the array is inserted into the scanner, where patterns of hybridization are detected. Data are collected as light emitted from the labels incorporated into the target, which becomes bound to the probe array. Probes that completely match the target generally produce stronger signals than those that have mismatches. The sequence and position of each probe on the array are known, and thus by complementarity, the identity of the target nucleic acid applied to the probe array can be determined.

Micro-arrays are prepared by selecting polynucleotide probes and immobilizing them to a solid support or surface. The probes may comprise DNA sequences, RNA sequences, copolymer sequences of DNA and RNA, DNA and/or RNA analogues, or combinations thereof. The probe sequences may be full or partial fragments of genomic DNA, or they may be synthetic oligonucleotide sequences synthesized either enzymatically in vivo, enzymatically in vitro (e.g., by PCR), or non-enzymatically in vitro.

The probe or probes used in the methods of the invention can be immobilized to a solid support or surface which may be either porous (e.g. gel), or non-porous. For example, the probes can be attached to a nitrocellulose or nylon membrane or filter covalently at either the 3′ or the 5′ end of the polynucleotide probe. The solid support may be a glass or plastic surface. In an aspect of the invention hybridization levels are measured to micro-arrays of probes consisting of a solid support on the surface of which are immobilized a population of polynucleotides.

In accordance with embodiments of the invention, a micro-array is provided comprising a support or surface with an ordered array of hybridization sites or “probes” each representing one of the markers described herein. The micro-arrays can be addressable arrays, and in particular positionally addressable arrays. Each probe of the array is typically located at a known, predetermined position on the solid support such that the identity of each probe can be determined from its position in the array. In preferred embodiments, each probe is covalently attached to the solid support at a single site.

Micro-arrays used in the present invention are preferably (a) reproducible, allowing multiple copies of a given array to be produced and easily compared with each other; (b) made from materials that are stable under hybridization conditions; (c) small, (e.g., between 1 cm² and 25 cm², between 12 cm² and 13 cm², or 3 cm²; and (d) comprise a unique set of binding sites that will specifically hybridize to the product of a single gene in a cell (e.g., to a specific mRNA, or to a specific cDNA derived therefrom). However, it will be appreciated that larger arrays may be used particularly in screening arrays, and other related or similar sequences will cross hybridize to a given binding site.

In accordance with an aspect of the invention, the micro-array is an array in which each position represents one of the KD Polynucleotides described herein. Each position of the array can comprise a DNA or DNA analogue based on genomic DNA to which a particular RNA or cDNA transcribed from a genetic marker can specifically hybridize. A DNA or DNA analogue can be a synthetic oligomer or a gene fragment. In an embodiment, probes representing each of the KD Polypeptides and KD Polynucleotides are present on the array. In a preferred embodiment, the array comprises at least 5, 10, 15, 20, 25, 30, 40, 50, 60, 70, 80, 90, 100, 125, 150, 175, 200, 225, 250, 275, or 300 genes comprising one or more KD Polynucleotides.

High-density oligonucleotide arrays containing thousands of oligonucleotides complementary to defined sequences, at defined locations on a surface can be produced using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). Using these methods oligonucleotides (e.g., 60-mers) of known sequence are synthesized directly on a surface such as a derivatized glass slide. The array produced may be redundant, with several oligonucleotide molecules per RNA.

Microarrays can be made by other methods including masking (Maskos and Southern, 1992, Nuc. Acids. Res. 20:1679-1684).

In an embodiment, microarrays of the present invention are produced by synthesizing polynucleotide probes on a support wherein the nucleotide probes are attached to the support covalently at either the 3′ or the 5′ end of the polynucleotide.

The invention provides micro-arrays comprising a disclosed marker set. In one embodiment, the invention provides a micro-array for distinguishing kidney disease samples comprising a positionally-addressable array of polynucleotide probes bound to a support, the polynucleotide probes comprising a plurality of polynucleotide probes of different nucleotide sequences, each of the different nucleotide sequences comprising a sequence complementary and hybridizable to a plurality of genes, the plurality consisting of at least 5, 10, 15, or 20 KD Polynucleotides. An aspect of the invention provides micro-arrays comprising at least 20, 25, 30, 35, 40, 45, 50, 75, 100, 125, 150, 175, 200, 225, 250, 275, or 300 genes including KD Polynucleotides.

The invention provides gene marker sets that distinguish kidney diseases and uses therefor. In an aspect, the invention provides a method for classifying a sample as associated with kidney disease comprising detecting a difference in the expression of a first plurality of genes relative to a control, the first plurality of genes consisting of one or more KD Polynucleotides. In another specific aspect, the control comprises nucleic acids derived from a pool of samples from individual term patients.

A micro-array can be used to monitor the time course of expression of one or more KD Polynucleotides in the array. This can occur in various biological contexts such as progression of kidney disease. Arrays are also useful for ascertaining differential expression patterns of KD Polynucleotides as described herein, and optionally other markers, in normal and abnormal samples. This may provide a battery of nucleic acids that could serve as molecular targets for diagnosis or therapeutic intervention.

Polypeptide Methods

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of kidney disease or stage or type of kidney disease in a subject may be determined by (a) contacting a sample from the subject with a binding agent; (b) detecting in the sample a level of KD Marker(s) that binds to the binding agent; and (c) comparing the level of KD Marker(s) with a predetermined standard or cut-off value.

In particular embodiments of the invention, the binding agent is an antibody. Antibodies specifically reactive with one or more KD Markers, or derivatives, such as enzyme conjugates or labelled derivatives, may be used to detect one or more KD Markers in various samples (e.g. biological materials). They may be used as diagnostic or prognostic reagents and they may be used to detect abnormalities in the level of expression of one or more KD Markers, or abnormalities in the structure, and/or temporal, tissue, cellular, or subcellular location of one or more KD Markers. Antibodies may also be used to screen potentially therapeutic compounds in vitro to determine their effects on kidney disease involving one or more KD Markers, and other conditions. In vitro immunoassays may also be used to assess or monitor the efficacy of particular therapies.

In an aspect, the invention provides a diagnostic method for monitoring or diagnosing kidney disease in a subject by quantitating one or more KD Markers in a biological sample from the subject comprising reacting the sample with antibodies specific for one or more KD Markers which are directly or indirectly labeled with detectable substances and detecting the detectable substances. In a particular embodiment of the invention, KD Markers are quantitated or measured.

In an aspect of the invention, a method for detecting kidney disease is provided comprising:
(a) obtaining a sample suspected of containing one or more KD Markers associated with kidney disease;
(b) contacting said sample with antibodies that specifically bind to the KD Markers under conditions effective to bind the antibodies and form complexes;
(c) measuring the amount of KD Markers present in the sample by quantitating the amount of the complexes; and
(d) comparing the amount of KD Markers present in the samples with the amount of KD Markers in a control, wherein a change or significant difference in the amount of KD Markers in the sample compared with the amount in the control is indicative of kidney disease.

In an embodiment, the invention contemplates a method for monitoring the progression of kidney disease in an individual, comprising:
(a) contacting antibodies which bind to one or more KD Markers with a sample from the individual so as to form complexes comprising the antibodies and one or more KD Markers in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of kidney disease in said individual.

The amount of complexes may also be compared to a value representative of the amount of the complexes from an individual not at risk of, or afflicted with, a kidney disease at different stages. A significant difference in complex formation may be indicative of advanced kidney disease, or an unfavourable prognosis.

In an embodiment of methods of the invention, VEGF-A, CXCR4, integrin β-1, PDGF-A, HIF1α and/or TGFβ are detected in samples and higher levels, in particular significantly higher levels compared to a control (normal) is indicative of kidney disease, in particular RPGN, more particularly pauci-immune RPGN.

In another embodiment of methods of the invention, pVHL is detected in samples and lower levels, in particular significantly lower levels compared to a control (normal) is indicative of kidney disease, in particular RPGN, more particularly pauci-immune RPGN.

In another embodiment of methods of the invention, VEGF-A, CXCR4, integrin β-1 PDGF-A, HIF1α and/or TGFβ are detected in samples and lower levels, in particular significantly lower levels compared to a control (normal) is indicative of kidney disease, in particular IgA nephropathy.

A particular embodiment of the invention comprises the following steps
(a) incubating a biological sample with first antibodies specific for one or more KD Markers which are directly or indirectly labeled with a detectable substance, and second antibodies specific for one or more KD Markers which are immobilized;
(b) detecting the detectable substance thereby quantitating KD Markers in the biological sample; and
(c) comparing the quantitated KD Markers with levels for a predetermined standard.

The standard may correspond to levels quantitated for samples from control subjects without kidney disease (normal), with a different stage of kidney disease, or from other samples of the subject. In an embodiment, increased levels of KD Markers as compared to the standard may be indicative of kidney disease. In another embodiment, lower levels of KD Markers as compared to the standard may be indicative of kidney disease.

Embodiments of the methods of the invention involve (a) reacting a biological sample from a subject with antibodies specific for one or more KD Markers which are directly or indirectly labelled with an enzyme; (b) adding a substrate for the enzyme wherein the substrate is selected so that the substrate, or a reaction product of the enzyme and substrate forms fluorescent complexes; (c) quantitating one or more KD Markers in the sample by measuring fluorescence of the fluorescent complexes; and (d) comparing the quantitated levels to levels obtained for other samples from the subject patient, or control subjects.

In another embodiment the quantitated levels are compared to levels quantitated for control subjects (e.g. normal) without kidney disease wherein an increase in KD Marker levels compared with the control subjects is indicative of kidney disease.

In further embodiment the quantitated levels are compared to levels quantitated for control subjects (e.g. normal) without kidney disease wherein a decrease in KD Marker levels compared with the control subjects is indicative of kidney disease.

Antibodies may be used in any known immunoassays that rely on the binding interaction between antigenic determinants of one or more KD Marker and the antibodies. Immunoassay procedures for in vitro detection of antigens in fluid samples are also well known in the art. [See for example, Paterson et al., Int. J. Can. 37:659 (1986) and Burchell et al., Int. J. Can. 34:763 (1984) for a general description of immunoassay procedures]. Qualitative and/or quantitative determinations of one or more KD Markers in a sample may be accomplished by competitive or non-competitive immunoassay procedures in either a direct or indirect format. Detection of one or more KD Markers using antibodies can be done utilizing immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

In an embodiment of the invention, an immunoassay for detecting more than one KD Marker in a biological sample comprises contacting binding agents that specifically bind to KD Markers in the sample under conditions that allow the formation of first complexes comprising a binding agent and KD Markers and determining the presence or amount of the complexes as a measure of the amount of KD Markers contained in the sample. In a particular embodiment, the binding agents are labeled differently or are capable of binding to different labels.

Binding agents (e.g. antibodies) may be used in immunohistochemical analyses, for example, at the cellular and sub-subcellular level, to detect one or more KD Markers, to localize them to particular cells and tissues, and to specific subcellular locations, and to quantitate the level of expression.

Immunohistochemical methods for the detection of antigens in tissue samples are well known in the art. For example, immunohistochemical methods are described in Taylor, Arch. Pathol. Lab. Med. 102:112 (1978). Briefly, in the context of the present invention, a tissue sample obtained from a subject suspected of having a kidney disease is contacted with antibodies, preferably monoclonal antibodies recognizing one or more KD Markers The site at which the antibodies are bound is determined by selective staining of the sample by standard immunohistochemical procedures. The same procedure may be repeated on the same sample using other antibodies that recognize one or more KD Markers. Alternatively, a sample may be contacted with antibodies against one or more KD Markers simultaneously, provided that the antibodies are labeled differently or are able to bind to a different label.

Antibodies specific for one or more KD Markers may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

One of the ways an antibody can be detectably labeled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steriod isomerase, yeast alcohol dehydrogenase, alpha-glycerophosphate, triose phosphate isomerase, asparaginase, glucose oxidase, and acetylcholine esterase.

A bioluminescent compound may also be used as a detectable substance. Bioluminescence is a type of chemiluminescence found in biological systems where a catalytic protein increases the efficiency of the chemiluminescent reaction. The presence of a bioluminescent molecule is determined by detecting the presence of luminescence. Examples of bioluminescent detectable substances are luciferin, luciferase and aequorin.

Indirect methods may also be employed in which the primary antigen-antibody reaction is amplified by the introduction of a second antibody, having specificity for the antibody reactive against one or more KD Markers. By way of example, if the antibody having specificity against one or more KD Markers is a rabbit IgG antibody, the second antibody may be goat anti-rabbit gamma-globulin labelled with a detectable substance as described herein.

Methods for conjugating or labelling the antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Cytochemical techniques known in the art for localizing antigens using light and electron microscopy may be used to detect one or more KD Markers. Generally, antibodies may be labeled with detectable substances and one or more KD Markers may be localised in tissues and cells based upon the presence of the detectable substances.

In the context of the methods of the invention, the sample, binding agents (e.g. antibodies specific for one or more KD Markers), or one or more KD Markers may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized antibody may be prepared by reacting the material with a suitable insoluble carrier using known chemical or physical methods, for example, cyanogen bromide coupling. An antibody may be indirectly immobilized using a second antibody specific for the antibody. For example, mouse antibody specific for a KD Marker may be immobilized using sheep anti-mouse IgG Fc fragment specific antibody coated on the carrier or support.

Where a radioactive label is used as a detectable substance, one or more KD Marker may be localized by radioautography. The results of radioautography may be quantitated by determining the density of particles in the radioautographs by various optical methods, or by counting the grains.

One or more KD Marker antibodies may also be indirectly labelled with an enzyme using ligand binding pairs. For example, the antibodies may be conjugated to one partner of a ligand binding pair, and the enzyme may be coupled to the other partner of the ligand binding pair. Representative examples include avidin-biotin, and riboflavin-riboflavin binding protein. In an embodiment, the antibodies are biotinylated, and the enzyme is coupled to streptavidin. In another embodiment, an antibody specific for KD Marker antibody is labeled with an enzyme.

Computer Systems

The analytic methods described herein can be implemented by use of computer systems and methods described below and known in the art. Thus the invention provides computer readable media comprising one or more KD Markers and/or KD Polynucleotides, and optionally other markers (e.g. markers of kidney disease). “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on one or more KD Markers and/or KD Polynucleotides, and optionally other markers.

A variety of data processor programs and formats can be used to store information on one or more KD Markers and/or KD Polynucleotides, and other markers on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention also provides in an electronic system and/or in a network, a method for determining whether a subject has kidney disease or a pre-disposition to kidney disease, comprising determining the presence or absence of one or more KD Markers and/or KD Polynucleotides, and optionally other markers, and based on the presence or absence of the one or more KD Markers and/or LI Polynucleotides, and optionally other markers, determining whether the subject has kidney disease, or a pre-disposition to kidney disease, and optionally recommending a procedure or treatment.

The invention further provides in a network, a method for determining whether a subject has kidney disease or a pre-disposition to kidney disease comprising: (a) receiving phenotypic information on the subject and information on one or more KD Markers and/or KD Polynucleotides, and optionally other markers associated with samples from the subject; (b) acquiring information from the network corresponding to the one or more KD Markers and/or KD Polynucleotides, and optionally other markers; and (c) based on the phenotypic information and information on the one or more KD Markers and/or KD Polynucleotides, and optionally other markers, determining whether the subject has kidney disease or a pre-disposition to kidney disease; and (d) optionally recommending a procedure or treatment.

The invention still further provides a system for identifying selected records that identify kidney disease. A system of the invention generally comprises a digital computer; a database server coupled to the computer; a database coupled to the database server having data stored therein, the data comprising records of data comprising one or more KD Markers and/or KD Polynucleotides, and optionally other markers, and a code mechanism for applying queries based upon a desired selection criteria to the data file in the database to produce reports of records which match the desired selection criteria.

In an aspect of the invention a method is provided for detecting cells or tissues associated with kidney disease using a computer having a processor, memory, display, and input/output devices, the method comprising the steps of:
(a) creating records of one or more KD Markers and/or KD Polynucleotides, and optionally other markers, identified in a sample suspected of containing KD Markers and/or KD Polynucleotides associated with kidney disease;
(b) providing a database comprising records of data comprising one or more KD Markers and/or KD Polynucleotides, and optionally other markers of kidney disease; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from cells or tissue that are associated with kidney disease.

The invention contemplates a business method for determining whether a subject has kidney disease or a pre-disposition to kidney disease comprising: (a) receiving phenotypic information on the subject and information on one or more KD Markers and/or KD Polynucleotides, and optionally other markers, associated with samples from the subject; (b) acquiring information from a network corresponding to one or more KD Markers and/or KD Polynucleotides, and optionally other markers; and (c) based on the phenotypic information, information on one or more KD Markers and/or KD Polynucleotides encoding the markers, and optionally other markers, and acquired information, determining whether the subject has kidney disease or a pre-disposition to a kidney disease; and (d) optionally recommending a procedure or treatment.

In an aspect of the invention, the computer systems, components, and methods described herein are used to monitor kidney disease or determine the stage or type of kidney disease.

Screening Methods

The invention also contemplates methods for evaluating putative agonists and antagonists for their ability to prevent, inhibit or reduce kidney disease, potentially contribute to kidney disease, or inhibit or enhance a type of kidney disease. Therefore, the invention provides a method for assessing the potential efficacy of a test agent for inhibiting kidney disease or onset of kidney disease in a patient, the method comprising comparing:
(a) levels of one or more KD Markers and/or KD Polynucleotides, and optionally other markers in a first sample obtained from a patient and exposed to the test agent; and
(b) levels of one or more KD Markers and/or KD Polynucleotides, and optionally other markers in a second sample obtained from the patient, wherein the sample is not exposed to the test agent, wherein a significant difference in the levels of expression of one or more KD Markers and/or KD Polynucleotides, and optionally the other markers, in the first sample, relative to the second sample, is an indication that the test agent is potentially efficacious for inhibiting kidney disease or onset of kidney disease in the patient.

The first and second samples may be portions of a single sample obtained from a patient or portions of pooled samples obtained from a patient.

In an aspect, the invention provides a method of selecting an agent for inhibiting kidney disease or onset of kidney disease in a patient comprising:
(a) obtaining a sample from the patient;
(b) separately maintaining aliquots of the sample in the presence of a plurality of test agents;
(c) comparing one or more KD Markers and/or KD Polynucleotides, and optionally other markers, in each of the aliquots; and
(d) selecting one of the test agents which alters the levels of one or more KD Markers and/or KD Polynucleotides, and optionally other markers in the aliquot containing that test agent, relative to other test agents.

Still another aspect of the present invention provides a method of conducting a drug discovery business comprising:
(a) providing one or more methods or assay systems for identifying agents that inhibit, prevent or reduce kidney disease, onset of kidney disease, or affect a stage or type of kidney disease in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the potential of a test compound to contribute to kidney disease or onset of kidney disease comprising:
(a) maintaining separate aliquots of cells or tissues from a patient with kidney disease in the presence and absence of the test compound; and
(b) comparing one or more KD Markers and/or KD Polynucleotides, and optionally other markers in each of the aliquots.

A significant difference between the levels of the markers in the aliquot maintained in the presence of (or exposed to) the test compound relative to the aliquot maintained in the absence of the test compound, indicates that the test compound possesses the potential to contribute to kidney disease or onset of kidney disease.

Kits

The invention also contemplates kits for carrying out the methods of the invention. Kits may typically comprise two or more components required for performing a diagnostic assay. Components include but are not limited to compounds, reagents, containers, and/or equipment.

The methods described herein may be performed by utilizing pre-packaged diagnostic kits comprising one or more specific KD Markers KD Polynucleotides, or binding agents (e.g. antibody) described herein, which may be conveniently used, e.g., in clinical settings to screen and diagnose patients and to screen and identify those individuals exhibiting a predisposition to developing kidney disease.

In an embodiment, a container with a kit comprises a binding agent as described herein. By way of example, the kit may contain antibodies or antibody fragments which bind specifically to epitopes of one or more KD Markers, and optionally other markers, antibodies against the antibodies labelled with an enzyme, and a substrate for the enzyme. The kit may also contain microtiter plate wells, standards, assay diluent, wash buffer, adhesive plate covers, and/or instructions for carrying out a method of the invention using the kit.

In an aspect of the invention, the kit includes antibodies or fragments of antibodies which bind specifically to an epitope of one or more of pVHL, VEGF-A, CXCR4, integrin β-1, PDGF-A, HIF1α and TGFβ and means for detecting binding of the antibodies to their epitope associated with kidney disease, either as concentrates (including lyophilized compositions), which may be further diluted prior to use or at the concentration of use, where the vials may include one or more dosages.

A kit may be designed to detect the level of polynucleotides encoding one or more KD Polynucleotides in a sample. In an embodiment, the polynucleotides encode one or more of pVHL, VEGF-A, CXCR4, integrin β-1, PDGF-A, HIF1α and TGFβ. Such kits generally comprise at least one oligonucleotide probe or primer, as described herein, that hybridizes to a KD Polynucleotide. Such an oligonucleotide may be used, for example, within a PCR or hybridization procedure.

The invention provides a kit containing a micoarray described herein ready for hybridization to target KD Polynucleotides, plus software for the data analysis of the results. The software to be included with the kit comprises data analysis methods, in particular mathematical routines for marker discovery, including the calculation of correlation coefficients between clinical categories and marker expression. The software may also include mathematical routines for calculating the correlation between sample marker expression and control marker expression, using array-generated fluorescence data, to determine the clinical classification of the sample.

The reagents suitable for applying the screening methods of the invention to evaluate compounds may be packaged into convenient kits described herein providing the necessary materials packaged into suitable containers.

The invention relates to a kit for assessing the suitability of each of a plurality of test compounds for inhibiting kidney disease or onset of kidney disease in a patient. The kit comprises reagents for assessing one or more KD Markers or KD Polynucleotides, and optionally a plurality of test agents or compounds.

The invention contemplates a kit for assessing the presence of cells and tissues associated with kidney disease or onset of kidney disease, wherein the kit comprises antibodies specific for one or more KD Markers, or primers or probes for KD Polynucleotides, and optionally probes, primers or antibodies specific for other markers associated with kidney disease.

Additionally the invention provides a kit for assessing the potential of a test compound to contribute to kidney disease. The kit comprises cells and tissues associated with kidney disease or onset of kidney disease and reagents for assessing one or more KD Markers, KD Polynucleotides, and optionally other markers associated with kidney disease.

Therapeutic Applications

Since VEGF-A, CXCR4, integrin B-1, PDGF-A, HIF1α, HIF2α, and TGFβ are increased or up-regulated in some kidney diseases, in particular RPGN, more particularly pauci-immune RPGN, they are targets for immunotherapy. Such immunotherapeutic methods include the use of antibody therapy, in vivo vaccines, and ex vivo immunotherapy approaches.

In one aspect, the invention provides VEGF-A, CXCR4, integrin B-1, PDGF-A, HIF1α and/or TGFβ antibodies that may be used systemically to treat kidney disease, in particular RPGN, more particularly pauci-immune RPGN. Thus, the invention provides a method of treating a patient susceptible to, or having a kidney disease, in particular RPGN, more particularly pauci-immune RPGN that expresses high levels of VEGF-A, CXCR4, integrin B-1, PDGF-A, HIF1α and/or TGFβ comprising administering to the patient an effective amount of an antibody which binds specifically to VEGF-A, CXCR4, integrin B-1, PDGF-A, HIF1α and/or TGFβ.

In the practice of the method of the invention, VEGF-A, CXCR4, integrin B-1, PDGF-A, HIF1α and/or TGFβ antibodies capable of specifically interacting with VEGF-A, CXCR4, integrin B-1, PDGF-A, HIF1α and/or TGFβ are administered in a therapeutically effective amount to patients whose kidney cells overexpress VEGF-A, CXCR4, integrin B-1, PDGF-A, HIF1α and/or TGFβ. The invention may provide a specific, effective and long-needed treatment for kidney diseases. The antibody therapy methods of the invention may be combined with other therapies.

Patients may be evaluated for the presence and levels of VEGF-A, CXCR4, integrin B-1, PDGF-A, HIF1α and/or TGFβ expression and overexpression in kidney cells, preferably using immunohistochemical assessments of kidney tissue, quantitative imaging, or other techniques capable of reliably indicating the presence and degree of VEGF-A, CXCR4, integrin B-1, PDGF-A, HIF1α and/or TGFβ expression. Immunohistochemical analysis of surgical specimens may be employed for this purpose.

VEGF-A, CXCR4, integrin B-1, PDGF-A, HIF1α and/or TGFβ antibodies useful in treating kidney disease include those that are capable of initiating a potent immune response. The activity of a particular antibody, or combination of antibodies, may be evaluated in vivo using a suitable animal model.

The methods of the invention contemplate the administration of single antibody cocktails as well as combinations, or “cocktails”, of different individual antibodies such as those recognizing different epitopes. Such cocktails may have certain advantages inasmuch as they contain antibodies which bind to different epitopes and/or exploit different effector mechanisms or combine directly cytotoxic antibodies with antibodies that rely on immune effector functionality. Such antibodies in combination may exhibit synergistic therapeutic effects. In addition, the administration of antibodies may be combined with other therapeutic agents. The antibodies may be administered in their “naked” or unconjugated form, or may have therapeutic agents conjugated to them.

Antibodies used in the practice of a method of the invention may be formulated into pharmaceutical compositions comprising a carrier suitable for the desired delivery method. Suitable carriers include any material which when combined with the antibodies retains the function of the antibody and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16.sup.th Edition, A. Osal., Ed., 1980).

Antibody formulations may be administered via any route capable of delivering the antibodies to the desired site. Routes of administration include, but are not limited to, intravenous, intraperitoneal, intramuscular, intradermal, and the like. Preferably, the route of administration is by intravenous injection. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration such as intravenous injection (IV), at an effective dose. Dosages will depend upon various factors generally appreciated by those of skill in the art, including the type of kidney disease and the severity or stage of the disease, the binding affinity and half life of the antibodies used, the degree of VEGF-A, CXCR4, INTEGRIN B-1, PDGF-A, HIF1α and/or TGFβ expression in the patient, the extent of circulating VEGF-A, CXCR4, INTEGRIN B-1, PDGF-A, HIF1α and/or TGFβ antigen, the desired steady-state antibody concentration level, frequency of treatment, and the influence of any therapeutic agents used in combination with the treatment method of the invention.

Daily doses may range from about 0.1 to 500 mg/kg. Doses in the range of 10-500 mg antibodies per week may be effective and well tolerated, although even higher weekly doses may be appropriate and/or well tolerated. A determining factor in defining the appropriate dose is the amount of a particular antibody necessary to be therapeutically effective in a particular context. Repeated administrations may be required to achieve inhibition or regression. Direct administration of antibodies is also possible and may have advantages in certain situations. Patients may be evaluated for serum VEGF-A, CXCR4, INTEGRIN B-1, PDGF-A, HIF1α and/or TGFβ in order to assist in the determination of the most effective dosing regimen and related factors. The KD Marker assay methods described herein, or similar assays, may be used for quantitating circulating VEGF-A, CXCR4, INTEGRIN B-1, PDGF-A, HIF1α and/or TGFβ levels in patients prior to treatment. Such assays may also be used for monitoring throughout therapy, and may be useful to gauge therapeutic success in combination with evaluating other parameters such as serum VEGF-A, CXCR4, INTEGRIN B-1, PDGF-A, HIF1α and/or TGFβ levels.

The invention further provides vaccines formulated to contain a VEGF-A, CXCR4, INTEGRIN B-1, PDGF-A, HIF1α and/or TGFβ protein or fragment thereof. The use in therapy of an antigen in a vaccine for generating humoral and cell-mediated immunity is well known. These methods can be practiced by employing a VEGF-A, CXCR4, INTEGRIN B-1, PDGF-A, HIF1α and/or TGFβ, or fragment thereof, or a polynucleotide encoding VEGF-A, CXCR4, INTEGRIN B-1, PDGF-A, HIF1α and/or TGFβ (i.e., KD Polynucleotide) and recombinant vectors capable of expressing and appropriately presenting these immunogens.

By way of example, viral gene delivery systems may be used to deliver a KD Polynucleotide. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA or fragment thereof introduced into the patient (e.g., intramuscularly) to induce a response.

Various ex vivo strategies may also be employed. One approach involves the use of cells to present VEGF-A, CXCR4, integrin B-1, PDGF-A, HIF1α and/or TGFβ antigen to a patient's immune system. For example, autologous dendritic cells which express MHC class I and II, may be pulsed with VEGF-A, CXCR4, integrin B-1, PDGF-A, HIF1α and/or TGFβ or peptides thereof that are capable of binding to MHC molecules, to thereby stimulate a patients' immune systems.

Anti-idiotypic antibodies can also be used in therapy as a vaccine for inducing an immune response to cells expressing a VEGF-A, CXCR4, integrin B-1, PDGF-A, HIF1α and/or TGFβ protein. The generation of anti-idiotypic antibodies is well known in the art and can readily be adapted to generate anti-idiotypic antibodies that mimic an epitope on a VEGF-A, CXCR4, integrin B-1, PDGF-A, HIF1α and/or TGF, protein.

Genetic immunization methods may be utilized to generate prophylactic or therapeutic humoral and cellular immune responses directed against cells expressing VEGF-A, CXCR4, integrin B-1, PDGF-A, HIF1α and/or TGFβ. Using VEGF-A, CXCR4, integrin B-1, PDGF-A, HIF1α and/or TGFβ encoding DNA molecules, constructs comprising DNA encoding a VEGF-A, CXCR4, integrin B-1, PDGF-A, HIF1α and/or TGFβ protein/immunogen and appropriate regulatory sequences may be injected directly into muscle or skin of an individual, such that the cells of the muscle or skin take-up the construct and express the encoded protein/immunogen. The protein/immunogen may be expressed as a cell surface protein or be secreted. Expression of the protein/immunogen results in the generation of prophylactic or therapeutic humoral and cellular immunity. Various prophylactic and therapeutic genetic immunization techniques known in the art may be used.

A KD Marker, and fragments thereof, and an agonist or antagonist may be used in the treatment of kidney disease in a subject. These polypeptides and agonists and antagonists may be formulated into compositions for administration to subjects suffering from kidney disease. Therefore, the present invention also relates to a composition comprising a KD Marker or a fragment thereof, an agonist, or antagonist and a pharmaceutically acceptable carrier, excipient or diluent. A method for treating or preventing kidney disease in a subject is also provided comprising administering to a patient in need thereof a KD Marker or a fragment thereof, an agonist, or antagonist, or a composition of the invention.

The invention further provides a method of inhibiting kidney disease in a patient comprising:
(a) obtaining a sample comprising diseased cells from the patient;
(b) separately maintaining aliquots of the sample in the presence of a plurality of test agents;
(c) comparing levels of pVHL, VEGF-A, CXCR4, integrin B-1, PDGF-A, HIF1α and/or TGFβ in each aliquot;
(d) administering to the patient at least one of the test agents which alters the levels of the pVHL, VEGF-A, CXCR4, integrin B-1, PDGF-A, HIF1α and/or TGFβ in the aliquot containing that test agent, relative to the other test agents.

In an embodiment, a test agent that decreases the levels of VEGF-A, CXCR4, integrin B-1, PDGF-A, HIF1α and/or TGFβ in an aliquot is administered to the patient. In another embodiment, a test agent that increases the levels of pVHL in an aliquot is administered to the patient.

The active substance may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment. The compositions of the invention may be administered concurrently, separately, or sequentially with other therapeutic agents or therapies.

The therapeutic activity of antibodies, compositions, agonists, and antagonists may be evaluated in vivo using a suitable animal model.

KD Polynucleotides associated with kidney disease can be turned off by transfecting a cell or tissue with vectors that express high levels of a desired KD Polynucleotide. Such constructs can inundate cells with untranslatable sense or antisense sequences. Even in the absence of integration into the DNA, such vectors may continue to transcribe RNA molecules until all copies are disabled by endogenous nucleases.

Vectors derived from retroviruses, adenovirus, herpes or vaccinia viruses, or from various bacterial plasmids, may be used to deliver KD Polynucleotides to a targeted organ, tissue, or cell population. Methods well known to those skilled in the art may be used to construct recombinant vectors that will express KD Polynucleotides such as antisense. (See, for example, the techniques described in Sambrook et al (supra) and Ausubel et al (supra).)

Methods for introducing vectors into cells or tissues include those methods discussed herein and which are suitable for in vivo, in vitro and ex vivo therapy. For example, delivery by transfection and by liposome are well known in the art.

Modifications of gene expression can be obtained by designing antisense molecules, DNA, RNA or PNA, to the regulatory regions of a KD Polynucleotide, i.e., the promoters, enhancers, and introns. Preferably, oligonucleotides are derived from the transcription initiation site, e.g. between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA are reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of KD Polynucleotides.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

The following non-limiting examples are illustrative of the present invention:

### EXAMPLE 1

### Summary

The von Hippel Lindau protein (pVHL) is a substrate recognition component of the E3 ubiquitin ligase complex that targets hypoxia inducible factors (HIFs) for proteasomal degradation. Mutations in the VHL gene are responsible for von Hippel Lindau disease characterized by renal cell carcinoma, vascular tumors of the central nervous system and pheochromocytoma [1]. Loss of pVHL and stabilization of HIFs leads to increased levels of factors implicated in tumor growth and metastasis including the chemokine receptor CXCR4 (fusin [2]), and vascular endothelial growth factor A (VEGF-A) [1,3]. To understand the role of pVHL in normal tissue function, the Cre-loxP system was used to generate kidney and lung-specific knock-out mice. Deletion of the VHL gene from podocytes and Type II pneumocytes leads to rapidly progressive glomerulonephritis (RPGN) and pulmonary hemorrhage, respectively. De novo expression of CXCR4 is seen in glomeruli from both mice and patients. The course of RPGN is dramatically improved in mice treated with a blocking antibody to CXCR4. Collectively, these results demonstrate that an intrinsic defect within vascular supporting cells alone is sufficient to cause small vessel vasculitis in mice and suggest novel molecular pathways for intervention in this devastating disease.

The following methods were used in the study described in the Example.

Methods

Generation of Podocyte-Specific VHL Knockout and CXCR4 Transgenic Mice

The podocin promoter was amplified from genomic murine DNA as described [Moeller et al, 2000] and cloned upstream of the NLS-Cre transgene and Cre-recombinase transgenic founder lines were generated as described [Grone et al, 2002]. Four individual founder lines were crossed with the Z/EG reporter strain [Moeller et al, 2004; Novak et al, 2000] to determine the degree and timing of Cre-mediated DNA excision in podocytes. One founder line was selected that gave 90% deletion of the floxed β-geo cassette in podocytes. The podocin-Cre or SP-C-Cre mice were bred with homozygous floxed VHL mice (VHL^{flox/flox}) (strain Vhlh^{tmIjae}, Jackson Labs) [Haase et al, 2001]. To generate homozygous floxed VHL-Cre recombinase mice, bitransgenic mice carrying both the podocin-Cre transgene and one floxed VHL allele were bred to homozygous floxed VHL mice.

The CXCR4 transgenic construct was generated by inserting a sequence-verified, full length 2-kb coding cDNA for CXCR4 (Open Biosystems, Livermore, Calif.) downstream of the 4.125-kb murine nephrin promoter in the pNXPRS vector as described elsewhere [Ermina et al, 2002; Natoli et al, 2002]. CXCR4 transgenic founder lines were generated as described [Joyner, 2000]. Three independent founder lines were identified and one with the most robust CXCR4 protein expression in podocytes chosen for further study.

All animal experimentation was conducted in accordance with the Canadian Guide for the Care and Use of Laboratory Animals.

Genotypic Analysis

Genomic DNA was isolated from tails of two-week-old transgenic mice and used for genotypic analysis as described. The Cre transgene was detected by PCR using the following primers: Cre 5′ 5′-ATGTCCAATTTACTGACCG3′ [SEQ ID NO.:12] and Cre 3′ 5′-CGCCGCATAACCAGTGAAAC 3[SEQ ID NO.:13], which amplified a band of approximately 300 bp. Conditions for the Cre PCR were as described [Ermina et al, 2002].

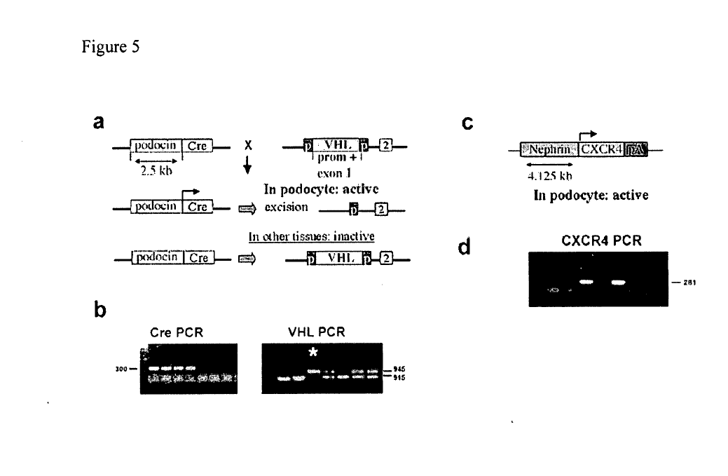

The floxed VHL gene was detected by PCR using the oligonucleotide primers olMR1555 (5′-CTCAGGTCATCTTCTGCAACC-3′) [SEQ ID NO.: 14] and olMR1556 (5TCTGTCTTGGCCTCCTGAGT-3′) [SEQ ID NO.: 15], which generate a 945-bp fragment for the floxed VHL allele and a 915-bp fragment for the wildtype VHL allele. Bands were separated on a 1.5% agarose gel (FIG. 5b).

The CXCR4 transgene was detected by PCR analysis using a primer in the nephrin promoter (5′-AACAGAAAAGCAGGGCACAC-3′) [SEQ ID NO.:16] and a second primer in the CXCR4 cDNA (5′-GTAGATGGTGGGCAGGAAGA-3′) [SEQ ID NO.:17]. Positive founders were identified by the presence of a 281-bp band (FIG. 5c).

Injection of 5-bromo-2-deoxyuridine (BrdU) and anti-CXCR4 Antibody

Three-week old VHL^{flox/flox}/Cre or VHL^{flox/+}/Cre mice were injected with 100 μg/g body weight BrdU (10 mg/ml) (Sigma, St. Louis, Mo., #B9285) in 0.9% NaCl solution. They received a second injection 15 hours later. Two hours after the second injection of BrdU, animals were sacrificed and kidneys were fixed in 4% paraformaldehyde overnight.

Each CXCR4 treatment group contained two VHL^{flox/flox}/Cre and 2 VHL^{flox/+}/Cre littermates. At 19 days after birth and then daily, rabbit anti-rat CXCR4 (Torry Pines Biolabs, Inc., Houston, Tex. #TP503) was administered to mice at a dose of 10 μg in 500 μl PBS by a single daily intraperitoneal injection as described [Petit et al, 2002]. Within each treatment group, one VHL^{flox/flox}/Cre mouse and one control mouse were given anti-rat CXCR4 while the second VHL^{flox/flox}/Cre mouse and control mouse were given 500 μl PBS (placebo). Urine was collected from mice daily at the same time each morning. Blood was collected at four weeks and at the time of sacrifice (seven weeks). A total of 20 mice were treated.

Phenotypic Analysis

Urine was collected passively in an Eppendorf tube from three week-old mice. A urine dipstick (Chemstrip 5L; Roche Diagnostics Corp., Indianapolis, Ind.) was used to detect the presence or absence of protein and red blood cells in the urine. The standard calorimetric assay was performed according to the manufacturer's instructions. In addition, 2 μl of urine from transgenic or control mice was placed in 18 μl of Laemmli buffer, boiled, and loaded on a 12% SDS-PAGE gel. An SDS-PAGE low-range protein standard (Bio-Rad Laboratories Inc., Hercules, Calif.) was loaded in the first lane of the gel.

Blood samples were taken with a heparinized capillary tube by femoral vein stab after warming. A total of 120 μl of blood was collected; creatinine, urea, and blood chemistry measurements were recorded using a Stat Profile M7 (Nova Biomedical Corp., Waltham, Mass.). The CBC (total blood count) was performed on a Coulter Counter (AcT diff; Beckman Coulter Canada, Ontario, Canada).

Statistical Analysis

Results are expressed as means. Student paired t test was used to analyze the difference between two groups. Values were regarded significant at p<0.05.

Histologic Analysis

Kidneys for histologic analysis were dissected, fixed in 10% formalin/PBS, and embedded in paraffin. Four μm thick sections were cut. Sections were stained with Periodic Acid Schiff stain (PAS) or hematoxylin and eosin (H&E) examined, and photographed with a DC200 Leica camera and Leica DMLB microscope (Leica Microsystems Inc., Deerfield, Ill.).

Laser Capture Microdissection (LCM)

One-half of a kidney was dissected from VHL^{flox/flox}/Cre mice and fixed in 4% PFA overnight, cryoprotected in 30% sucrose overnight, embedded in Tissue Tek OCT 4583 (Sakura. Finetek USA Inc., Torrance, Calif.) and snap frozen. Ten micron cryosections were prepared on blood smear slides (Surgipath), stained with toluidine blue and used for LCM. Pure cell populations from mouse glomeruli, renal tubules or cellular crescents were obtained using the AutoPix™ Automated Laser Capture Microdissection System according to manufacturer's instructions.

DNA was extracted from the LCM sample using the PicoPure™ DNA Extraction Kit protocol. Digested DNA samples were stored at −20° C. until PCR amplification. DNA samples from LCM were amplified by PCR using the following primers: VHL-FW5 primer 5′-CTG GTA CCC ACG AAA CTG TC-3′ [SEQ ID NO.: 18] (upstream of 5loxP). VHL-FWD primer 5′-CTA GGC ACC GAG CTT AGA GGT TTG CG-3[SEQ ID NO.: 19] (upstream of 2^{nd} loxP in intron 1). VHL-RVS primer 5′-CTG ACT TCC ACT GAT GCT TGT CAC AG-3′ [SEQ ID NO.:20] (downstream of 2^{nd} loxP in intron 1). The VHL 2-loxP allele is represented by a 460-bp band, the 1-loxP allele by a 350-bp band. The three primers together were used to assess Cre-mediated DNA excision in 2-loxP homozygotes.

Five μl of digested DNA was subjected to 42 cycles of PCR in a volume of 50 μl containing 20 pmol VHL-FW5 primer, 20 pmol VHL-FWD primer, 40 pmol VHL-RVS primer, 1 μl of Taq DNA polymerase, 3 μl of 25 mM MgCl₂, 5 μl of 10×PCR buffer, 1 μl of 10 mM dNTP and 27 μl autoclaved ddH₂O. PCR conditions were 94° C. for two min 30 sec, 94° C. for 50 sec, 57° C. for 50 sec, 72° C. for one min and last extension at 72° C. for five min. PCR products were electrophoresed on a 2% agarose gel.

RNA was similarly extracted from LCM samples and cDNA was reverse transcribed from purified RNA using the Superscript™ First-Strand Synthesis System for RT-PCR (Invitrogen, Life Technologies, Carlsbad, Calif.), and stored at −20° C. until use for real-time PCR. cDNA samples from LCM were quantitated using the ABI 7900 (Applied Biosystems, Foster City, Calif.) according to manufacturer's instructions, using the following murine primers: CXCR4-FWD primer 5′-CAG AGG CCA AGG AAA CTG CT-3′ [SEQ ID NO.: 21], CXCR4-REV primer 5′-CTG ACG TCG GCA AAG ATG AA-3′ [SEQ ID NO.: 22], 18S-FWD primer 5′-AGG AAT TGA CGG AAG GGC AC-3′ [SEQ ID NO.: 23], 18S-REV primer 5′-GGA CAT CTA AGG GCA TCA CA-3′ [SEQ ID NO.: 24].

Two μl of cDNA was subjected to real-time PCR on the ABI 7900 (Applied Biosystems, Foster City, Calif.) in a volume of 10 μl containing 900 nM CXCR4-FWD, 900 nM CXCR4-REV, 150 nM 18S-FWD, and 150 nM 18-S primers. SYBR® Green Master Mix was used for all PCR reactions, and universal cycling conditions were followed according to the ABI standard method as follows: initial hold of ten minutes at 95° C. followed by 40 cycles at 95° C. for 15 seconds and 60° C. for 60 seconds. For quantitative analyses, each VHL^{flox/flox}/Cre cDNA sample was compared with a sample from a control littermate following the delta delta CT (ΔΔ CT) method (ABI, User Bulletin #2), and all samples were normalized to 18S.

In Situ Hybridization and Immunohistochemistry

Kidneys were dissected from mice on postnatal day six and at three weeks, four weeks, or seven weeks of age. Kidneys were washed briefly in RNase-free PBS and fixed overnight in DEPC-treated 4% paraformaldehyde. These tissues were then placed in 30% sucrose for 12-24 hours, embedded in Tissue-Tek OCT and snap frozen. Ten-micron tissue samples were cut on a Leica Jung cryostat (model CM3050; Leica Microsystems Inc.) and transferred to Superfrost microscope slides (Fisher Scientific Co., Pittsburgh, Pa., USA). The slides were stored at −20° C. until needed. Digoxigenin-labeled probes were prepared according to the Roche Molecular Biochemicals protocol (Roche Molecular Biochemicals, Mannheim, Germany). Probes used for in situ analysis were nephrin [Wong et al, 2000], and VEGF-A [Ermina et al, 2003].

Primary antibodies used were ZO-1 in a 1:10 dilution (J. Miner, St. Louis, Mo.), BrdU 1:10 dilution (cat. No 1 585 860; Roche Diagnostics GmbH, Mannheim, Germany); CXCR4 monoclonal antibody, 1:100 dilution (Cedarlane Laboratories Ltd., Ontario, Canada); GFP antibody, 1:2000 dilution (Molecular Probes, Eugene, Oreg.); HIF-1α rabbit polyclonal antibody, 1:100 dilution, (R&D Systems, Catalog Number AB1536); fibrinogen, 1:100 dilution (DakoCytomation, Denmark).

Secondary antibodies used for anti-BrdU and CXCR4 were Cy3-conjugated AffiniPure Donkey Anti-mouse IgG 1:500 dilution (Jackson ImmunoResearch laboratories Inc., # 715-165-151); secondary for ZO-1 was FITC conjugated affiniPure goat anti-Rat IgG 1:10 dilution (H & L, #112-095-003); secondary for GFP and Hif-1α was anti-rabbit IgG biotinylated antibody, 1:200 dilution (ABC kit, Vector laboratories, Burlingame, Calif.). PCNA staining was performed with the ZYMED PCNA Staining Kit (ZYMED Laboratories Inc., Cat. No. 93-1143 South San Francisco) according to manufacturer's instructions.

Human studies were performed on 6 μM cryosections taken from tissue biopsies with primary antibodies to CXCR4, 1:50 dilution (Abcam, Cambridge, UK), and monoclonal mouse synaptopodin, prediluted (clone G1D4, Progen, Heidelberg, Germany). Secondary antibodies used for anti-CXCR4 were Alexa Fluor 546 anti-rabbit (Molecular Probes, Invitrogen) and for anti-synaptopodin were Alexa Fluor 488 goat anti-mouse (Molecular Probes, Invitrogen). Glomerular Isolation and Microarray Analysis were performed as described [Takemoto et al, 2002; Cui et al, 2005] using the 45K mouse Affymetrix Chips (MOE430) (Santa Clara, Calif.). All studies were performed at the microarray facility, The Centre for Applied Genomics, The Hospital for Sick Children Toronto. Briefly, glomeruli were isolated from 4 week wildtype or VHL^{flox/flox}/Cre mice. RNA was isolated and probes for microarray hybridization were generated using the Affymetrix two-cycle Kit. In vitro transcription was performed with the Affymetrix IVT kit; miscanning was performed with the Affymetrix GeneChip Scanner 3000. Three independent experiments were performed.

Transfection of Podocyte Cell Lines:

A conditionally immortalized mouse podocyte cell line was isolated from H-2 KbtsA58 transgenic mice kidneys (ImmortoMouse®; Charles River Labs) as previously described (Mundel & Shankland, 2002; Somlo & Mundel, 2000). In these mice, a temperature-sensitive SV40 large T cell antigen (tsA58 Tag) is controlled by interferon-γ inducible H-2 Kb promoter. When grown under growth permissive conditions (33° C. with 50 units/ml mouse INF-γ) podocytes proliferate; when grown under growth restrictive conditions (37° C. in the absence of mouse INF-γ) podocytes stop proliferating and differentiate. Podocytes were characterized as previously described [Davies et al, 1982].

Transient transfection of pc3-myc-x-4-his-119s vector expressing active CXCR4 or pc3-myc-x-4-his-WT vector expressing wild type CXCR4 was performed on podocytes growth restricted to day 12. Each vector (10 μg) was combined with 25 ul of n-fect transfection reagent (Neuromics, Edina, Minn., USA) and applied to 170,000 cells for 72 hours. Cell Titer 96 Non-Radioactive Cell Proliferation Assay, or MTT assay (Promega, Madison, Wis., USA) was used to determine cell number as per manufacturers directions.

Podocytes were infected with murine stem cell virus puromycin (pMSCVpuro) containing either enhanced green fluorescent protein (empty vector) or myc-CXCR4. Briefly, each vector was transfected into the Phoenix viral producing cell line. Supernatants containing shed virus were applied to growth permissive podocytes for 3 infection cycles. Infected podocytes were selected for puromycin resistance at 3 μg/ml for 72 hours and resistant cells were expanded. Infected podocytes were re-plated on growth restrictive conditions. Proliferation was measured by MTT assay at days 2, 4, 6 and 8 of growth restriction. Infected podocytes were fixed in methanol at the same time points and immunostained with a mouse monoclonal antibody to Ki-67 (BD Pharmingen, San Diego, Calif., USA) followed by a fluorescent sheep anti-mouse secondary (Jackson ImmunoResearch Laboratories, Inc., West Grove, Pa., USA). Positive staining nuclei were quantified as a percentage of the total nuclei.

To confirm transfection and infection of podocytes, 10 μg of whole cell lysate was separated under reduced conditions on 15% SDS-polyacrylamide gels and transferred to PVDF membrane (Immobilon-P; Millipore, Bedford, Mass., USA). Membranes were incubated with a rabbit antibody against c-myc (Alpha Diagnostic International, Inc., San Antonio, Tex., USA) overnight at 4° C., followed by incubation with an alkaline phosphatase-conjugated anti-rabbit IgG antibody (Promega, Madison, Wis., USA) at room temperature for 60 min. Detection was performed using the chromagen 5-bromo-4-chloro-3-indolyl phosphate/nitro blue tetrazolium (Sigma).

CXCR4 and VHL Target Gene Expression in Human Renal Biopsies.

Human kidney biopsies, obtained in a multicenter study for renal gene expression analysis (the European renal cDNA consortium, ERCB), were processed as described [Cohen et al, 2002]. Informed consent was obtained according to the respective local ethical committee guidelines. Histologies were stratified by the reference pathologists of the ERCB: IgA glomerulonephritis (n=15), cANCA-positive rapidly progressive glomerulonephritis (n=9), and control biopsies from non-neoplastic parts of tumor-nephrectomies (n=5). Real-time RT-PCR was performed as previously described [Cohen et al, 2002]. The following sequences of oligonucleotide primers (300 nM) and probes (100 nM) were used for CXCR4: sense primer 5′-GGC CGA CCT CCT CTT TGT C-3′ [SEQ ID NO.:25], antisense primer 5′-CAA AGT ACC AGT TTG CCA CGG-3′ [SEQ ID NO.:26] fluorescence labelled probe (FAM) 5′-ACG CTT CCC TTC TGG GCA GTT GAT C-3′ [SEQ ID NO.:27] (obtained from Applied Biosystems, Weiterstadt, Germany). Pre-developed TaqMan assay reagent was used for the internal standard Glycerin-aldehyde-3-phosphate-dehydrogenase (GAPDH) and HIF1α, Integrin-β1 and TGF-β1. Expression levels are shown as ratios to GAPDH and expressed as ratio to the mean of controls. Quantification of the given templates was performed according to the standard curve method. All measurements were performed in duplicate; controls consisting of bi-distilled H₂O were negative in all runs.

Description of Study

A study was designed to genetically delete the product of the von Hippel Lindau Gene (pVHL) selectively from podocytes. pVHL is a component of the E3 ubiquitin ligase that targets proteins for degradation in the proteasome. Loss of pVHL leads to stabilization of the hypoxia inducible factor alpha subunits and subsequent upregulation hypoxia-response downstream genes. Although pVHL and HIFs have not been directly implicated in RPGN, a number of HIF target genes are known to be increased in RPGN including TNF alpha, TNF alpha receptor and VEGF-A [Timoshanko et al, 2003; Suga et al, 2001]. Furthermore, a number of cases of RPGN have been reported in patients with renal cell carcinoma, a disease associated with mutations in the VHL gene [Sommer et al, 1998, Kagan et al, 1993, Norris et al. In this study, it is shown that loss of pVHL selectively from podocytes is sufficient to generate cellular crescents and the clinical picture of pauci-immune RPGN in mice, in the absence of ANCA antibodies. In contrast to the widely accepted paradigm that vascular injury in vasculitis and specifically RPGN is the result of circulating factors, in the genetic model herein, this injury is initiated by podocytes that sit on the ‘other side’ of the endothelium away from the circulation and reside in the urinary space.

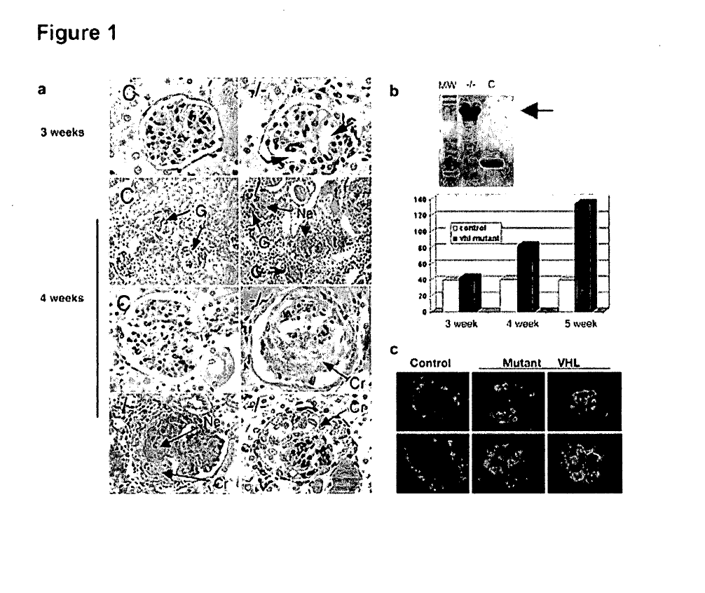

To selectively delete VHL from renal podocytes, a podocyte-specific Cre recombinase murine line (pod-Cre) was bred with mice that carry a floxed wild-type VHL allele (FIG. 5a, 5b) [Haase et al, 2001]. Cre-mediated DNA excision generates a null VHL allele through deletion of the promoter and 1st exon [Haase et al, 2001. Mice of all genotypes were born in the expected Mendelian frequency and appeared well until 4 weeks of age, when they developed an explosive onset of renal disease with hematuria, proteinuria and renal insufficiency (FIG. 1a, b). These mice rapidly succumbed to renal failure by 7 weeks of age. Histologic exam of their kidneys at 4 weeks showed crescentic glomerulonephritis with prominent segmental fibrin deposition and fibrinoid necrosis (FIG. 1a, c). Notably, immune deposits were not observed on immunofluorescent examination. Taken together, these are features characteristic of pauci-immune RPGN.

To determine the clinical course of the disease, mice and their kidneys were examined at earlier timepoints. At one week of age, the glomeruli of VHL flox/flox/Pod-Cre mice were histologically normal. By three weeks of age, proteinuria (1 g/L) was detected in the urine of all VHL flox/flox/Pod-Cre mice, but the mice were active and appeared healthy and their renal function was not different from controls. On histologic exam, the glomeruli were normal with the exception of dilated capillary loops (FIG. 1a). However, just 1 week later, 100% of VHL^{flox/flox}/Pod-Cre mice developed an acute onset of disease, similar to the presentation of RPGN observed in patients. Given the interest in circulating factors in development of this disease, the presence of circulating ANCA antibodies was looked for in the mice at the height of their disease but none were found (n=3). Aside from the vascular inflammation or glomerulitis, crescent formation is a striking and consistent finding in glomeruli of patients with RPGN and in these transgenic mice. It is currently accepted that crescentic cells are derived from parietal epithelium and influxing inflammatory cells such as macrophages [Couser, 2004]. More recently, lineage tagging showed that podocytes also contribute to crescent formation in an immune-mediated mouse model of anti-GBM RPGN [Moeller et al, 2004].

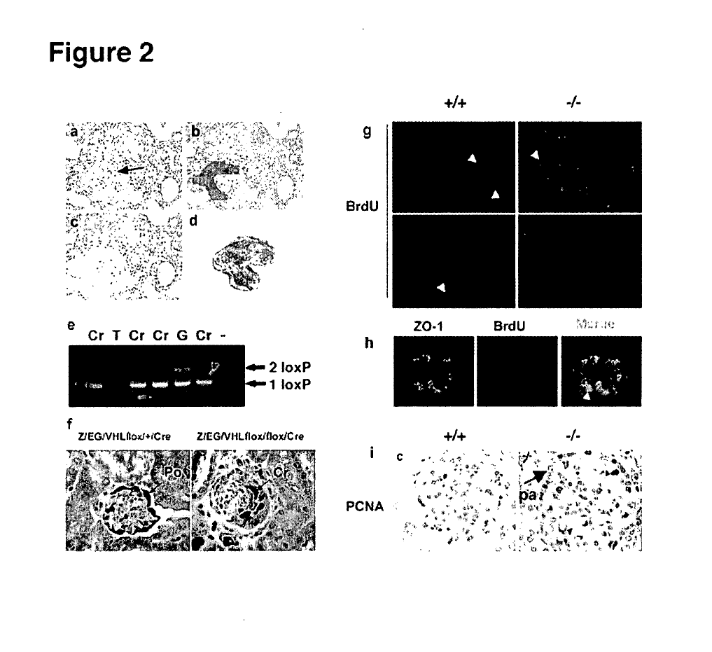

Given the experimental design, the proliferating cells that form the crescents in the model of RPGN were speculated to originate from the podocyte cell lineage. Laser capture microdissection was used to isolate genomic DNA from the cellular crescents (FIG. 2a-e). PCR analysis clearly demonstrates excision of the floxed VHL allele from crescentic DNA but not tubular DNA confirming that these cells originate from podocytes that express the Cre transgene. To confirm this finding, the podocyte cell lineage was tagged with a green fluorescent protein (GFP) reporter transgene [Novak et al, 2000] that is activated only upon Cre-mediated DNA excision; all cells within the crescent express GFP demonstrating that they originated from the podocyte lineage (FIG. 2f). To exclude an environmental effect due to the ‘conventional status’ of the mouse facility, the mice were rederived in a pathogen-free barrier facility and no difference was found in phenotype. Taken together, these results demonstrate that an intrinsic defect in glomeruli is sufficient to initiate RPGN.

It is widely accepted that terminally differentiated podocytes cannot proliferate and, to date, no genetic model exists where podocytes are ‘switched back on’ to divide. To determine whether podocytes re-enter the cell cycle, undergo proliferation, and therefore generate the cellular crescents in the model described herein, bromodeoxyuridine labeling (BrdU) was performed (FIG. 2g). Double immunostaining with the podocyte-restricted marker ZO-1 shows that in the early stages of disease (prior to crescent formation), podocytes are proliferating (FIG. 2h). PCNA (proliferating cell nuclear antigen) staining shows that glomerular epithelial cell proliferation continues within the crescent at four weeks and at this stage, also includes parietal epithelial cells (FIG. 2i).

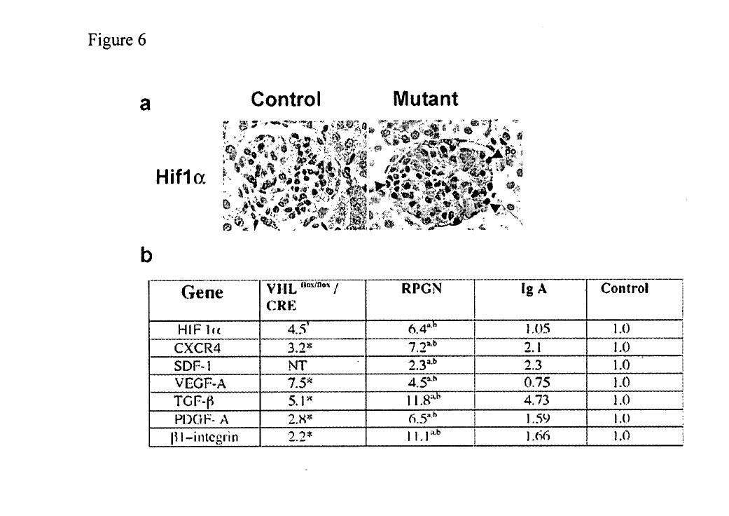

To characterize the molecular response in glomeruli of the transgenic mice and to identify candidate targets for intervention in this disease, gene expression profiling was performed between glomeruli isolated from mutant and wildtype littermates. The best-studied target(s) for pVHL are the hypoxia inducible factor alpha (HIF-α) subunits. Loss of pVHL stabilizes both the HIF1 and 2 alpha subunits and leads to increased expression of hypoxia-response genes including VEGF-A, CXCR4, TGF-β, PDGF-A and Hif1-α itself. Accordingly, both HIF1-α and HIF2-α protein levels were increased in podocytes from VHL^{flox/flox}//Pod-Cre mice (FIG. 6a) and microarray analysis confirmed that expected VHL downstream target genes as well as known RPGN genes were increased in glomeruli isolated from VHL^{flox/flox}/Pod-Cre compared to glomeruli from wildtype littermates (FIG. 6b, 7).

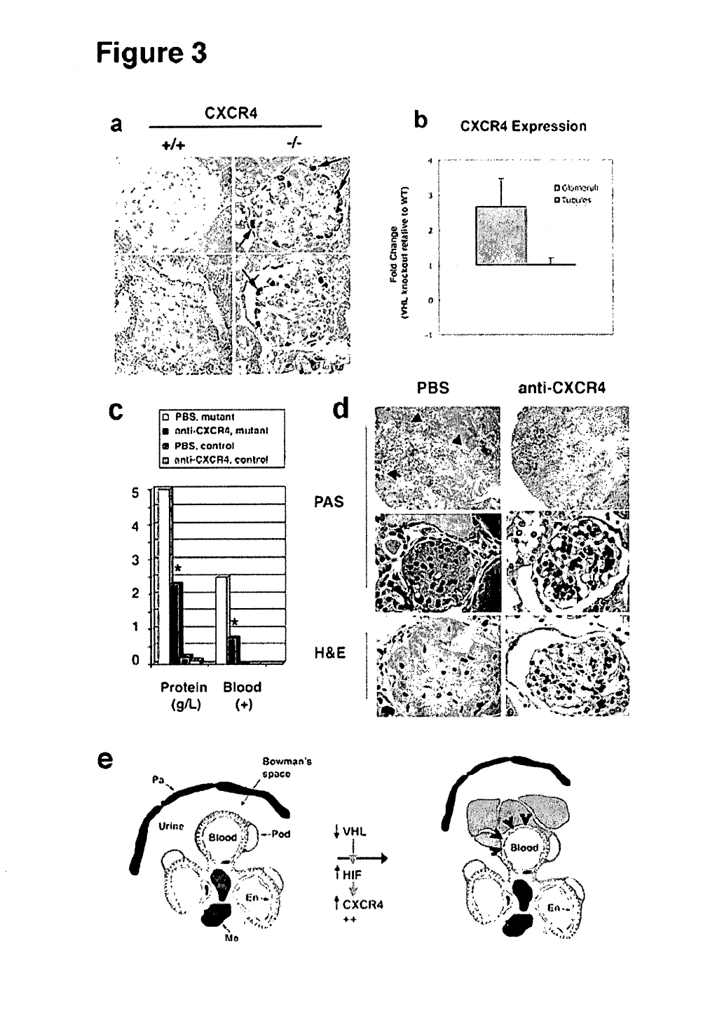

Given that CXCR4 is involved in the migratory and proliferative capacity of both cancer and hematopoietic cells [Staller et al, 2003], it was identified as a possible candidate to be functionally involved in the phenotypic switch observed in podocytes from VHL mutants. FIG. 3a demonstrates de novo expression of CXCR4 within podocytes of mutant mice compared to absent expression in glomeruli of wildtype littermates. LCM and realtime PCR confirmed a 2.8-fold increase in glomerular CXCR4 mRNA that was absent from tubules (FIG. 3b). Mesangial cells, which sit between the glomerular capillary loops, express the only known ligand for CXCR4— stromal-derived factor-1 (SDF-1) [16]. In situ hybridization and microarray analysis confirmed that SDF-1 mRNA is expressed at high levels in mesangial cells during glomerular development and persists in both wildtype and mutant adult glomeruli.

To determine if inhibition of CXCR4 may be a therapeutic option in RPGN, mutant and control littermates at nineteen days of age were injected with a blocking antibody to CXCR4 [17]. This time point was chosen because it precedes the onset of crescent formation and is the earliest date that the genotype of the mice could be determined. The onset of nephrotic-range proteinuria was delayed in treated vs. untreated mutant mice by 5 to 7 days and the severity of glomerular disease was markedly diminished as determined by the degree of proteinuria (p<0.025), hematuria (p<0.02) and glomerular pathology (FIG. 3c, d). Mutant mice treated with PBS alone exhibited 100% mortality at 7 weeks of age compared with 0% in the group receiving anti-CXCR4 therapy. Taken together, these results are consistent with a model in which upregulation of the chemokine receptor CXCR4 contributes to the phenotypic switch in podocytes permitting them to proliferate and form the cellular crescents that surround the glomerular tuft (FIG. 3e).

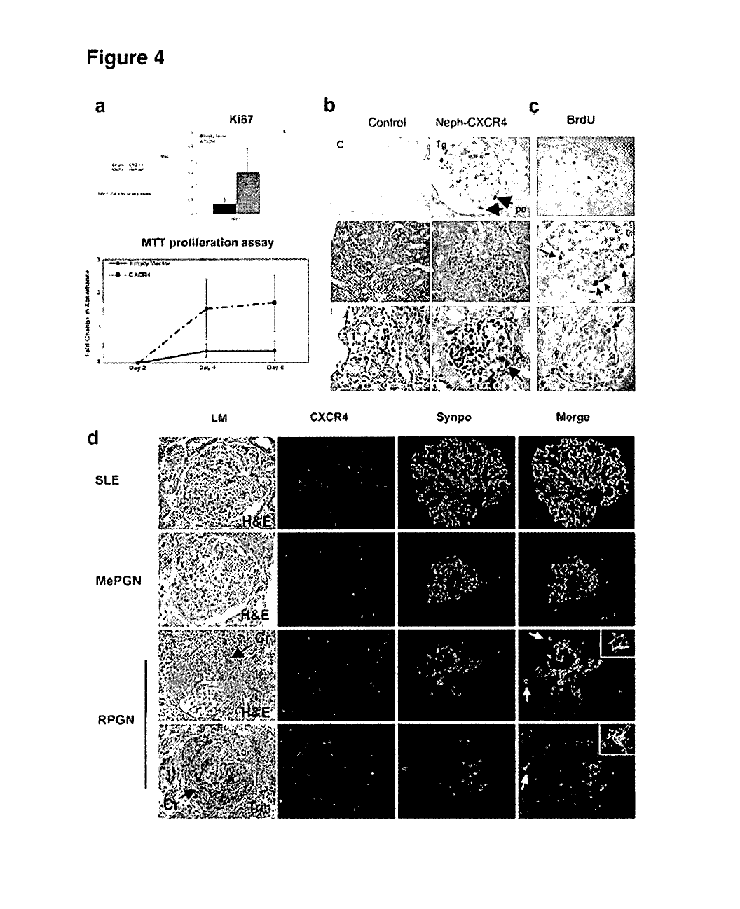

In support of this conclusion, fully differentiated immortalized podocytes that have been transfected with a constitutively active version of the CXCR4 receptor [Zhang et la, 2002] show increased proliferation. A significantly higher proportion of CXCR4-positive podocytes expressed Ki67 (FIG. 4a), a marker of entry into S-phase of the cell cycle, at the onset of cell proliferation (day 2) and cell number was increased as measured by MTT assay (FIG. 4a).

To directly test this model in vivo, transgenic mice that express CXCR4 selectively within their podocytes (CXCR4Pod) were generated. The data show that de novo expression of CXCR4 alone is sufficient to cause glomerular disease and proliferation of podocytes in vivo. By 6 months of age, CXCR4Pod mice were found to have blood (1+) and protein (1 g/L) in their urine, diagnostic of glomerular disease. Light microscopy showed that 80%-100% of glomeruli from CXCR4Pod mice were markedly enlarged (FIG. 4a) (n=3) compared to control glomeruli. Mice were pulsed with BrdU; immunostaining confirmed the presence of proliferating cells within glomeruli with positive staining in podocytes (FIG. 4b). A portion of glomeruli had focal crescents or crescent-like structures (FIG. 4b). These results suggest that CXCR4 is both required and sufficient for podocyte proliferation but that other VHL targets are required for full blown RPGN as observed in VHL^{flox/flox}/Pod-Cre mice.

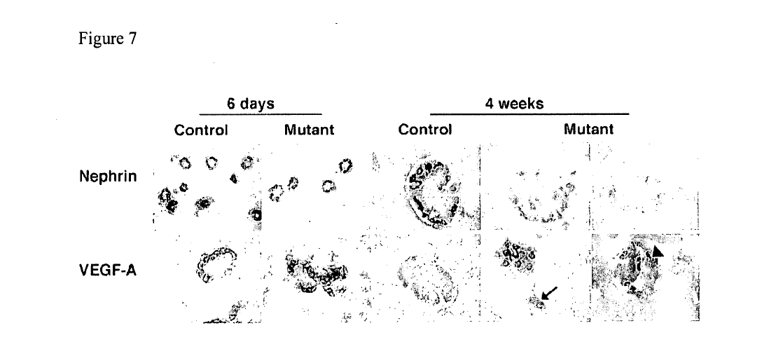

To determine if stabilization of HIFs and upregulation of downstream targets also occur in glomeruli of patients with pauci-immune RPGN, real time PCR analysis and immunostaining was performed (FIG. 6b, c & d). Strikingly, the same ‘expression fingerprint’ of VHL target genes was found including a 7.2-fold increase (p<0.015) in CXCR4 (FIG. 6). Immunostaining confirmed that CXCR4 is markedly increased in glomeruli from patients with ANCA+ pauci-immune RPGN compared to patients with other renal diseases (FIG. 4d). Conversely, synaptopodin—a marker for differentiated podocytes—is decreased in RPGN; this loss of podocyte differentiation also occurs in VHL mutant mice (FIG. 7). Currently, there are no methods available to identify ‘de-differentiated’ podocytes in crescents of patients and may explain why they have escaped detection in biopsy specimens. Despite this, a few cells within the crescents co-stain for both CXCR4 and synaptopodin (FIG. 4a), consistent with the lineage tagging experiments in mice that show crescentic cells originate from podocytes and express CXCR4.

In summary, this study demonstrated that pVHL is required in the podocyte to maintain glomerular integrity. Loss of pVHL permits terminally differentiated podocytes to re-enter the cell cycle. Upregulation of CXCR4 and rescue of the phenotype in VHL^{flox/flox}/pod-Cre transgenic mice with CXCR4 blocking antibodies, show that this pathway is functionally important. The absence of any systemic or circulating perturbation in the model, demonstrates that intrinsic defects alone in the glomerulus are sufficient to initiate crescent formation and renal vasculitis in mice. Together, the results provide a new paradigm for the pathogenesis of small vessel vasculitis and glomerular disease where the inciting endothelial injury occurs from the ‘outside-in’.

Thus, Applicants have shown that loss of a gene from a single intrinsic cell population within the glomerulus is sufficient to generate cellular crescents and the clinical picture of pauci-immune RPGN. Furthermore, this cell population is on the ‘other side’ of the endothelium and resides in the urinary space.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

### FULL CITATIONS FOR REFERENCES REFERRED TO IN THE SPECIFICATION

1. Mundel, P. & Shankland, S. J. Podocyte biology and response to injury. J Am Soc Nephrol 13, 3005-3015 (2002).
2. Somlo, S. & Mundel, P. Getting a foothold in nephrotic syndrome. Nat Genet 24, 333-335 (2000).
3. Davies, D. J., Moran, J. E., Niall, J. F. & Ryan, G. B. Segmental necrotising glomerulonephritis with antineutrophil antibody: possible arbovirus aetiology? Br Med J (Clin Res Ed) 285, 606 (1982).
4. Falk, R. J., Terrell, R. S., Charles, L. A. & Jennette, J. C. Anti-neutrophil cytoplasmic autoantibodies induce neutrophils to degranulate and produce oxygen radicals in vitro. Proc Natl Acad Sci USA 87, 4115-9 (1990).
5. Xiao, H. et al. Antineutrophil cytoplasmic autoantibodies specific for myeloperoxidase cause glomerulonephritis and vasculitis in mice. J Clin Invest 110, 955-63 (2002).
6. Timoshanko, J. R., Sedgwick, J. D., Holdsworth, S. R. & Tipping, P. G. Intrinsic renal cells are the major source of tumor necrosis factor contributing to renal injury in murine crescentic glomerulonephritis. J Am Soc Nephrol 14, 1785-93 (2003).
7. Suga, S. et al. Vascular endothelial growth factor (VEGF121) protects rats from renal infarction in thrombotic microangiopathy. Kidney Int 60, 1297-1308 (2001).
8. Sommer, M. et al. Rapidly progressive glomerulonephritis in a patient with advanced renal cell carcinoma. Nephrol Dial Transplant 13, 2107-9 (1998).
9. Kagan, A., Sinay-Trieman, L., Czernobilsky, B., Barzilai, N. & Bar-Khayim, Y. Is the association between crescentic glomerulonephritis and renal cell carcinoma coincidental? Nephron 65, 642-3 (1993).
10. Norris, J. H., Leeds, J. & Jeffrey, R. F. P-ANCA positive renal vasculitis in association with renal cell carcinoma and prolonged hydralazine therapy. Ren Fail 25, 311-4 (2003).
11. Haase, V. H., Glickman, J. N., Socolovsky, M. & Jaenisch, R. Vascular tumors in livers with targeted inactivation of the von Hippel-Lindau tumor suppressor. Proc Natl Acad Sci USA 98, 1583-8 (2001).
12. Couser, B. Crescent Formation. www.uptodate.com, Review (2004).
13. Moeller, M. J. et al. Podocytes populate cellular crescents in a murine model of inflammatory glomerulonephritis. J. Am. Soc. Nephrol 15, 61-67 (2004).
14. Novak, A., Guo, C., Yang, W., Nagy, A. & Lobe, C. G. Z/EG, a double reporter mouse line that expresses enhanced green fluorescent protein upon Cre-mediated excision. Genesis 28, 147-155 (2000).
15. Staller, P. et al. Chemokine receptor CXCR4 downregulated by von Hippel-Lindau tumour suppressor pVHL. Nature 425, 307-311 (2003).
16. Grone, H. J. et al. Spatial and temporally restricted expression of chemokines and chemokine receptors in the developing human kidney. J Am Soc Nephrol 13, 957-67 (2002).
17. Bezzi, P. et al. CXCR4-activated astrocyte glutamate release via TNFalpha: amplification by microglia triggers neurotoxicity. Nat Neurosci 4, 702-710 (2001).
18. Zhang, W. B. et al. A point mutation that confers constitutive activity to CXCR4 reveals that T140 is an inverse agonist and that AMD3100 and ALX40-4C are weak partial agonists. J Biol Chem 277, 24515-21 (2002).
19. Moeller, M. J., Kovari, I. A. & Holzman, L. B. Evaluation of a new tool for exploring podocyte biology: mouse Nphsl 5′ flanking region drives LacZ expression in podocytes. J. Am. Soc. Nephrol 11, 2306-2314 (2000).
20. Belteki, G. et al. Conditional and inducible transgene expression in mice through the combinatorial use of Cre-mediated recombination and tetracycline induction. Nucleic Acids Res 33, e51 (2005).
21. Eremina, V., Wong, M. A., Cui, S., Schwartz, L. & Quaggin, S. E. Glomerular-specific gene excision in vivo. J. Am. Soc. Nephrol 13, 788-793 (2002).
22. Natoli, T. A. et al. A Mutant Form of the Wilms' Tumor Suppressor Gene WTI Observed in Denys-Drash Syndrome Interferes with Glomerular Capillary Development. J Am Soc Nephrol 13, 2058-2067 (2002).
23. Joyner, A. L. Gene Targeting, A Practical Approach, 293 p (Oxford University Press, New York, 2000).
24. Petit, I. et al. G-CSF induces stem cell mobilization by decreasing bone marrow SDF-1 and up-regulating CXCR4. Nat Immunol 3, 687-694 (2002).
25. Fischer, S. et al. Dynamic changes in apoptotic and necrotic cell death correlate with severity of ischemia-reperfusion injury in lung transplantation. Am J Respir Crit Care Med 162, 1932-9 (2000).
26. Wong, M. A., Cui, S. & Quaggin, S. E. Identification and characterization of a glomerularspecific promoter from the human nephrin gene. Am. J. Physiol. Renal Physiology 279, F1027-F1032 (2000).
27. Eremina, V. et al. Glomerular-specific alterations of VEGF-A expression lead to distinct congenital and acquired renal diseases. J. Clin. Invest 111, 707-716 (2003).
28. Takemoto, M. et al. A new method for large scale isolation of kidney lomeruli from mice. American Journal of Pathology 161, 799-805 (2002).
29. Cui, S. et al. Rapid Isolation of Glomeruli Coupled with Gene Expression Profiling Identifies Downstream Targets in Pod1 Knockout Mice. JASN, In Press 16 (2005).
30. Cohen, C. D., Frach, K., Schlondorff, D. & Kretzler, M. Quantitative gene expression analysis in renal biopsies: a novel protocol for a high-throughput multicenter application. Kidnet Int 61, 133-40 (2002).

1. (canceled)

2. A method for screening a subject for kidney disease, the method comprising comparing:
(a) levels of one or more of pVHL VEGF-A, CXCR4, integrin β-1, PDGF-A, HIF1α and TGFβ in a sample from the subject; and
(b) normal levels of pVHL VEGF-A, CXCR4, integrin β-1, PDGF-A, HIF1α and TGFβ in a control sample, wherein a significant difference in levels of pVHL VEGF-A, CXCR4, integrin β-1, PDGF-A, HIF1α and TGFβ relative to the corresponding normal levels, is indicative of kidney disease.

3. A method as claimed in claim 2 comprising:
(a) contacting a biological sample obtained from a subject with one or more binding agent that specifically binds topVHL, VEGF-A, CXCR4, integrin β-1, PDGF-A, HIF1α and TGFβ or parts thereof; and
(b) detecting in the sample amounts of pVHL VEGF-A, CXCR4, integrin β-1, PDGF-A, HIF1α and TGFβ that bind to the binding agents, relative to a predetermined standard or cut-off value, and therefrom determining the presence or absence of the kidney disease in the subject.

4. A method as claimed in claim 3 wherein the binding agent is an antibody.

5. A method for screening a subject for kidney disease comprising (a) obtaining a biological sample from a subject; (b) detecting in proteins extracted from the sample the amount of one or more of pVHL VEGF-A, CXCR4, integrin β-1, PDGF-A, HIF1α and TGFβ; and (c) comparing the amount of pVHL VEGF-A, CXCR4, integrin β-1, PDGF-A, HIF1α and TGFβ detected to a predetermined standard, where detection of levels of pVHL. VEGF-A, CXCR4, integrin β-1, PDGF-A, HIF1α and TGFβ different than that of a standard is indicative of kidney disease.

6. A method of claim 5 wherein the levels of VEGF-A, CXCR4, integrin β-1, PDGF-A, HIF1α and TGFβ are significantly higher compared to the standard and are indicative of RPGN, in particular pauci-RPGN.

7. A method of claim 5 wherein the level of pVHL is significantly lower compared to the standard and is indicative of RPGN, in particular pauci-RPGN.

8. A method of claim 5 wherein the levels of VEGF-A, CXCR4, integrin β-1, PDGF-A, HIF1α and TGFβ are significantly lower compared to the standard and are indicative of IgA nephropathy.

9. A method as claimed in claim 2 wherein the sample is obtained from tissues, extracts, cell cultures, cell lysates, lavage fluid, or physiological fluids.

10. A method according to claim 2 wherein the presence or absence of one or more polynucleotide markers encoding pVHL VEGF-A, CXCR4, integrin β-1, PDGF-A, HIF1α and TGFβ are detected in a sample from the subject and detected amounts are related to the presence of kidney disease.

11. A method as claimed in claim 10 wherein the polynucleotide detected is mRNA.

12. A method of claim 11 wherein the polynucleotide is detected by
(a) contacting the sample with oligonucleotides that hybridize to the polynucleotides; and
(b) detecting in the sample levels of nucleic acids that hybridize to the polynucleotides relative to a predetermined standard or cut-off value, and therefrom determining the presence or absence of kidney disease in the subject.

13. A method as claimed in claim 12 wherein the mRNA is detected using an amplification reaction.

14. A method as claimed in claim 13 wherein the amplification reaction is a polymerase chain reaction employing oligonucleotide primers that hybridize to the polynucleotides, or complements of such polynucleotides.

15. A method as claimed in claim 13 wherein the mRNA is detected using a hybridization technique employing oligonucleotide probes that hybridize to the polynucleotides or complements of such polynucleotides.

16. A method as claimed in claim 15 wherein the mRNA is detected by (a) isolating mRNA from the sample and combining the mRNA with reagents to convert it to cDNA; (b) treating the converted cDNA with amplification reaction reagents and primers that hybridize to the polynucleotides, to produce amplification products; (d) analyzing the amplification products to detect an amount of mRNA encoding one or more of the markers; and (e) comparing the amount of mRNA to an amount detected against a panel of expected values for normal tissue derived using similar primers.

17. (canceled)

18. (canceled)

19. (canceled)

20. (canceled)

21. (canceled)

22. (canceled)

23. (canceled)

24. (canceled)

25. (canceled)

26. A method of treating kidney disease in a subject comprising administering to a subject in need thereof an antagonist of CXCR4.

27. A method according to claim 26 wherein the kidney disease is RPGN.

28. A method according to claim 27 wherein the kidney disease is pauci-immune RPGN.

29. A kit for carrying out a method as claimed in claim 2.

30. (canceled)

31. (canceled)


SEQUENCE LISTING (28 sequences)

SEQ ID NO: 1 - protein, 213 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 2 - protein, 172 aa, Homo sapiens
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRVYTLKERCLQVVRSLVKPENYRRLDIVRSLYEDLEDHPNVQKDLERLTQERIAHQRMGD

SEQ ID NO: 3 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 4 - protein, 356 aa, Homo sapiens
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 5 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 6 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 7 - protein, 735 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGII

SEQ ID NO: 8 - protein, 798 aa, Homo sapiens
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 9 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 10 - protein, 211 aa, Homo sapiens [sequence omitted]

SEQ ID NO: 11 - protein, 196 aa, Homo sapiens
MRTLACLLLLGCGYLAHVLAEEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 12 - DNA, 19 bp, Homo sapiens
atgtccaatttactgaccg

SEQ ID NO: 13 - DNA, 20 bp, Homo sapiens
cgccgcataaccagtgaaac

SEQ ID NO: 14 - DNA, 21 bp, Homo sapiens
ctcaggtcatcttctgcaacc

SEQ ID NO: 15 - DNA, 20 bp, Homo sapiens
tctgtcttggcctcctgagt

SEQ ID NO: 16 - DNA, 20 bp, Homo sapiens
aacagaaaagcagggcacac

SEQ ID NO: 17 - DNA, 20 bp, Homo sapiens
gtagatggtgggcaggaaga

SEQ ID NO: 18 - DNA, 20 bp, Homo sapiens
ctggtacccacgaaactgtc

SEQ ID NO: 19 - DNA, 26 bp, Homo sapiens
ctaggcaccgagcttagaggtttgcg

SEQ ID NO: 20 - DNA, 26 bp, Homo sapiens
ctgacttccactgatgcttgtcacag

SEQ ID NO: 21 - DNA, 20 bp, Homo sapiens
cagaggccaaggaaactgct

SEQ ID NO: 22 - DNA, 20 bp, Homo sapiens
ctgacgtcggcaaagatgaa

SEQ ID NO: 23 - DNA, 20 bp, Homo sapiens
aggaattgacggaagggcac

SEQ ID NO: 24 - DNA, 20 bp, Homo sapiens
ggacatctaagggcatcaca

SEQ ID NO: 25 - DNA, 19 bp, Homo sapiens
ggccgacctcctctttgtc

SEQ ID NO: 26 - DNA, 21 bp, Homo sapiens
caaagtaccagtttgccacgg

SEQ ID NO: 27 - DNA, 25 bp, Homo sapiens
acgcttcccttctgggcagttgatc

SEQ ID NO: 28 - protein, 92 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKFKM